(19) United States
(12) Patent Application Publication
(10) Pub. No.: US 2025/0001172 A1
(43) Pub. Date: Jan. 2, 2025

(54) Methods, Systems, Devices And Components for Miniaturized Implantable Gastric Electrical Stimulators and Corresponding Medical Electrical Leads and Electrodes

(71) Applicant: Enterra Medical, Inc., St. Louis Park, MN (US)

(72) Inventors: John Swoyer, Blaine, MN (US); Peter Sommerness, Minneapolis, MN (US); Warren Lee Starkebaum, Plymouth, MN (US); Jason James Hamann, Blaine, MN (US); Barry Mitchell Yomtov, Marblehead, MA (US)

(21) Appl. No.: 18/674,491

(22) Filed: May 24, 2024

Related U.S. Application Data
(60) Provisional application No. 63/468,703, filed on May 24, 2023.

(51) Int. Cl. A61N 1/36; A61N 1/02; A61N 1/05; A61N 1/372; A61N 1/375; A61N 1/378

(52) U.S. Cl. CPC A61N 1/36007; A61N 1/0509; A61N 1/37217; A61N 1/375; A61N 1/378; A61N 1/025

(57) ABSTRACT

Described and disclosed herein are various embodiments of methods, systems, devices and components relating to miniaturized gastric electrical stimulators (GESs) configured for implantation in a patient's stomach.

This application is related to, and claims priority and other benefits from, U.S. Provisional Patent Application Ser. No. 63/468,703 entitled “Methods, Systems, Devices And Components for the Treatment of Nausea And Vomiting with Gastric Electrical Stimulation” to Starkebaum et al. filed on May 24, 2023 (hereafter “the '703 provisional patent application”), and claims priority and other benefits therefrom. The '703 provisional patent application is hereby incorporated by reference herein, in its entirety, to provide continuity of disclosure. This application is also related to U.S. Utility patent application Ser. No. ______ entitled “Electrical Stimulation Methods, Systems, Devices and Components for Monitoring, Diagnosing and Treating Gastric Disorders” to Starkebaum et al. filed on even date herewith (May 24, 2024), the entirety of which hereby incorporated by reference herein.

### FIELD OF THE INVENTION

The present invention relates to systems, devices, components and methods associated with miniaturized implantable gastric electrical stimulators and corresponding medical electrical leads and electrodes.

### BACKGROUND

Systems, devices, components and methods associated with temporary gastric electrical stimulators and corresponding medical electrical leads and electrodes employed to treat various gastric disorders in patients typically require patients to have a wired unwieldy and uncomfortable device running through the patient's nose or mouth to an implantable gastric stimulator. What is needed are systems, devices, components and methods for temporary gastric electrical stimulation that are less bulky, less uncomfortable, and less disruptive to a patient.

### SUMMARY

In some embodiments, there is provided a temporary or permanent gastric stimulator comprising a stimulation module and at least one stimulation electrode operably connected thereto and associated therewith, the stimulation module forming or forming a portion of an implantable pulse generator (IPG), wherein at least portions of the stimulator are configured to fit within a first space or volume formed in a submucosal layer of a patient's stomach using an endoscopic device, wherein the endoscopic device is configured to be advanced into the first space or volume to form a second space or tunnel in the submucosal layer or to form an extension of the first space in the submucosal layer, and further wherein at least portions of the stimulator are configured to be implanted in at least a portion of the second space or tunnel of the submucosal layer of the patient's stomach, or to be implanted in the extension of the first space of the submucosal layer of the patient's stomach.

Such a temporary or permanent gastric stimulator may further comprise one or more of: (i) wherein the temporary or permanent gastric stimulator is further configured to, over a first predetermined period of time, generate first electrical stimulation signals in the IPG, the first electrical stimulation signals having one or more frequencies ranging between about 2 Hz and about 120 Hz, one or more pulse widths ranging between about 100 μsec. and about 10 msec., one or more amplitudes ranging between about 0.1 mA and about 20 mA; (ii) wherein the temporary or permanent gastric stimulator is further configured to provide, over at least a portion of the first predetermined period of time, the first electrical stimulation signals through at least one medical electrical lead and one or more electrodes thereof to a portion of the stomach of the patient, and over a second predetermined period of time, generate second electrical stimulation signals in the IPG, the second electrical stimulation signals having one or more of frequencies, pulse widths and amplitudes less than one or more of the corresponding frequencies, pulse widths and amplitudes of the first electrical stimulation signals; (iii) wherein the temporary or permanent gastric stimulator is further configured to provide, over at least a portion of the second predetermined period of time, the second electrical stimulation signals through the at least one medical electrical lead and the one or more electrodes thereof to the portion of the stomach of the patient; (iv) wherein the temporary or permanent gastric stimulator is further configured to provide, as one or more of the frequencies, pulse widths, and amplitudes of the electrical stimulation signals provided to the patient continue to be successively reduced, determining one or more of the frequency, pulse width, and amplitude parameters of the electrical stimulation signal wherein efficacy in treating the one or more gastrointestinal disorders of the patient is reduced or lost; (v) wherein the temporary or permanent gastric stimulator is further configured to provide, on the basis of the frequency, pulse width, and amplitude parameters determined to be associated with reduced or lost efficacy in treating the one or more gastrointestinal disorders of the patient, generating and providing to the patient chronic electrical stimulation signals having one or more of increased frequency, pulse width, and amplitude parameters compared to the frequency, pulse width, and amplitude parameters determined to be associated with substantially reduced or lost efficacy in treating the one or more gastrointestinal disorders of the patient; (vi) wherein the one or more gastrointestinal disorders of the patient include at least one of nausea, vomiting, early satiety, postprandial fullness, and abdominal pain and the method results in more rapid and accurate programming and determination of gastric stimulation parameters for the patient compared to conventional gastric stimulation programming techniques or methods; (vii) wherein the implantable stimulation module and the at least one electrode are contained in, or each form a portion of, a capsule or housing; (viii) wherein the temporary or permanent gastric stimulator further comprises electrical stimulation electronics and a power source associated therewith; (ix) wherein the temporary or permanent gastric stimulator further comprises communication electronics configured to permit hard-wired or wireless communication and control or programming thereof from a programmer or controller; (x) wherein the stimulation module comprises at least one return or ground electrode; (xi) wherein the at least one stimulation electrode forms a portion of a medical electrical lead, the medical electrical lead being operably connected to the implantable stimulation module and configured to deliver electrical stimulation signals from the module to the lead; (xii) wherein the lead comprises multiple stimulation electrodes; (xiii) wherein the electrodes are at least one of unipolar, bipolar and multi-polar; (xiv) wherein the lead comprises at least one return or ground electrode; (xv) wherein a biodegradable or releasable link is disposed between at least a portion of the lead and the stimulation module, and at least one of the lead and the stimulation module is configured to be released from attachment to or positioning within the tunnel, the first space, the second space, or the submucosal layer, and then to pass harmlessly through the patient's digestive system after the biodegradable link has dissolved or the link has been released after a predetermined period of time has passed or upon receipt of a command by the stimulator from an external communication device; (xvi) wherein the temporary or permanent gastric stimulator further or one or more portions thereof such as a lead portion comprises at least one fixation member or feature configured to affix the stimulator or portion thereof to the submucosal layer; (xvii) wherein the fixation member or feature comprises one or multiple ones of a tine, a helical fixation wire, a staple, and a fixation pin; (xviii) wherein the temporary or permanent gastric stimulator further comprises one or more of a rechargeable battery, a primary battery, and a power source; (xix) wherein the stimulator is implanted using one or more G POEM steps, techniques or methods; (xx) further comprising the temporary or permanent gastric stimulator or one or more portions thereof, being configured to be passed safely through the patient's digestive tract after being released from the stomach by the temporary or permanent gastric stimulator or one or more portions thereof being released or through the action of a biodegradable link dissolving.

In other embodiments, there is provided a method of implanting a temporary or permanent gastric stimulator in a stomach of a patient, the temporary or permanent gastric stimulator comprising a stimulation module and at least one stimulation electrode operably connected thereto and associated therewith, the stimulation module forming or forming a portion of an implantable pulse generator (IPG), wherein at least portions of the stimulator are configured to fit within a first space or volume formed in a submucosal layer of a patient's stomach using an endoscopic device, wherein the endoscopic device is configured to be advanced into the first space or volume to form a second space or tunnel in the submucosal layer or to form an extension of the first space in the submucosal layer, and further wherein at least portions of the stimulator are configured to be implanted in at least a portion of the second space or tunnel of the submucosal layer of the patient's stomach, or to be implanted in the extension of the first space of the submucosal layer of the patient's stomach, the method comprising: (a) advancing the endoscopic device into the first space or volume to form the second space or tunnel in the submucosal layer or to form an extension of the first space in the submucosal layer, and (b) using the endoscopic device, implanting the stimulator in at least a portion of the second space or tunnel of the submucosal layer of the patient's stomach or implanting the stimulator in the extension of the first space of the submucosal layer of the patient's stomach.

Such a method may further comprise one or more of: (i) the temporary or permanent gastric stimulator generating over a first predetermined period of time first electrical stimulation signals, the first electrical stimulation signals having one or more frequencies ranging between about 2 Hz and about 120 Hz, one or more pulse widths ranging between about 100 μsec. and about 10 msec., and one or more amplitudes ranging between about 0.1 mA and about 20 mA; (ii) the temporary or permanent gastric stimulator further providing, over at least a portion of the first predetermined period of time, the first electrical stimulation signals through at least one medical electrical lead and one or more electrodes thereof to a portion of the stomach of the patient, and over a second predetermined period of time, generates second electrical stimulation signals in the IPG, the second electrical stimulation signals having one or more of frequencies, pulse widths and amplitudes less than one or more of the corresponding frequencies, pulse widths and amplitudes of the first electrical stimulation signals; (iii) the temporary or permanent gastric stimulator providing over at least a portion of the second predetermined period of time, the second electrical stimulation signals through the at least one medical electrical lead and the one or more electrodes thereof to the portion of the stomach of the patient; (iv) the temporary or permanent gastric stimulator providing, as one or more of the frequencies, pulse widths, and amplitudes of the electrical stimulation signals provided to the patient continues to be successively reduced, determining one or more of the frequency, pulse width, and amplitude parameters of the electrical stimulation signal wherein efficacy in treating the one or more gastrointestinal disorders of the patient is reduced or lost; (v) the temporary or permanent gastric stimulator providing, on the basis of the frequency, pulse width, and amplitude parameters determined to be associated with reduced or lost efficacy in treating the one or more gastrointestinal disorders of the patient, generating and providing to the patient chronic electrical stimulation signals having one or more of increased frequency, pulse width, and amplitude parameters compared to the frequency, pulse width, and amplitude parameters determined to be associated with substantially reduced or lost efficacy in treating the one or more gastrointestinal disorders of the patient; (vi) the one or more gastrointestinal disorders of the patient including at least one of nausea, vomiting, early satiety, postprandial fullness, and abdominal pain and the method results in more rapid and accurate programming and determination of gastric stimulation parameters for the patient compared to conventional gastric stimulation programming techniques or methods; (vii) the implantable stimulation module and the at least one electrode being contained in, or each form a portion of, a capsule or housing; (viii) the temporary or permanent gastric stimulator comprising electrical stimulation electronics and a power source associated therewith; (ix) the temporary or permanent gastric stimulator further comprising communication electronics configured to permit hard-wired or wireless communication and control or programming thereof from a programmer or controller; (x) the stimulation module comprising at least one return or ground electrode; (xi) the at least one stimulation electrode forming a portion of a medical electrical lead, the medical electrical lead being operably connected to the implantable stimulation module and configured to deliver electrical stimulation signals from the module to the lead; (xii) the lead comprising multiple stimulation electrodes; (xiii) the electrodes being at least one of unipolar, bipolar and multi-polar; (xiv) the lead comprising at least one return or ground electrode; (xv) a biodegradable or releasable link being disposed between at least a portion of the lead and the stimulation module, and at least one of the lead and the stimulation module is configured to be released from attachment to or positioning within the tunnel, the first space, the second space, or the submucosal layer, and then to pass harmlessly through the patient's digestive system after the biodegradable link has dissolved or the link has been released after a predetermined period of time has passed or upon receipt of a command by the stimulator from an external communication device; (xvi) the temporary or permanent gastric stimulator or one or more portions thereof such as a lead portion comprising at least one fixation member or feature configured to affix the stimulator or portion thereof to the submucosal layer; (xvii) the fixation member or feature comprising one or multiple ones of a tine, a helical fixation wire, a staple, and a fixation pin; (xviii) the temporary or permanent gastric stimulator comprising one or more of a rechargeable battery, a primary battery, and a power source; (xix) the stimulator being implanted using one or more G POEM steps, techniques or methods; and (xx) the temporary or permanent gastric stimulator or one or more portions thereof, being configured to be passed safely through the patient's digestive tract after being released from the stomach by the temporary or permanent gastric stimulator or one or more portions thereof being released or through the action of a biodegradable link dissolving.

Further embodiments are disclosed herein or will become apparent to those skilled in the art after having read and understood the claims, specification and drawings hereof.

### BRIEF DESCRIPTION OF THE DRAWINGS

Different aspects of the various embodiments will become apparent from the following specification, drawings and claims in which:

FIGS. 1 through 6 show various embodiments of electrical/electronic block diagrams for implantable pulse generators (IPGs), gastric electrical stimulators (GESs) 10 and/or temporary gastric electrical stimulators (temporary GESs) 10, and various components associated therewith;

FIG. 7 shows one embodiment of a method for treating patients with severe or moderate gastrointestinal (GI) symptoms alone or accompanied by different dysmotility disorders;

FIG. 8 shows an electrogastrogram (EGG) A in combination with electrical stimulation pulses B and C;

FIG. 9 shows illustrative fundic responses in patients;

FIG. 10 shows illustrative heart rates in a patient over a 24-hour period;

FIG. 11 shows one embodiment of a method/algorithm 500 configured to provide greater specificity for automatically adjusting electrical stimulation parameters of an IPG or GES during a nocturnal cycle;

FIG. 12 shows one embodiment of a method/algorithm 600 configured to provide greater specificity for adapting the adjustment of a nocturnal schedule for an IPG or GES to a patient's circadian schedule;

FIG. 13 shows one embodiment of a method/algorithm 700 for an IPG or GES to sense and detect events associated with gastric disorders.;

FIG. 14 shows one embodiment of a method/algorithm 800 configured to detect, record and employ pattern recognition or other similar analytical techniques to improve gastric disorder event detection in an IPG or GES;

FIG. 15 illustrates a pH meter readout device positioned on a patient;

FIG. 16 shows one embodiment of a capsule-shaped GES/IPG;

FIG. 17 shows another view of the capsule-shaped GES/IPG of FIG. 16;

FIG. 18 shows another embodiment of a capsule-shaped GES/IPG;

FIG. 19 shows yet another embodiment of a capsule-shaped GES/IPG;

FIG. 20 shows one embodiment of an implantable medical electrical lead 1000 configured for attachment to an implantable IPG/GES/capsule;

FIG. 21 shows another embodiment of an implantable medical electrical lead 1000 configured for attachment to an implantable IPG/GES/capsule;

FIG. 22 shows yet another embodiment of an implantable medical electrical lead 1000 configured for attachment to an implantable IPG/GES/capsule;

FIG. 23 shows one embodiment of an implantable IPG/GES/capsule configured for implantation in a patient via endoscopic means;

FIG. 24 shows another embodiment of an implantable IPG/GES/capsule configured for implantation in a patient via endoscopic means;

FIG. 25 shows one illustrative embodiment of an electrical/electronic block diagram of an implantable IPG/GES/capsule;

FIGS. 26A and 26B show examples of GES burst cycling;

FIGS. 27 through 30 show various embodiments of implantable IPGs/GESs/capsules configured for implantation in a patient, and

FIGS. 31A through 33E show various embodiments of implantable IPGs/GESs/capsules being positioned in and affixed to patients' stomachs.

The drawings are not necessarily to scale. Like numbers refer to like parts or steps throughout the drawings.

### DETAILED DESCRIPTIONS OF SOME EMBODIMENTS

First we describe various embodiments of gastric electrical stimulation systems, devices, components and methods, and block diagrams associated with some embodiments thereof. Next we describe various embodiments of methods, systems, devices and components associated with treating nausea and vomiting using gastric electrical stimulation techniques. Then we describe various embodiments of methods, systems, devices and components associated with temporary and permanent gastric stimulation. It is also contemplated that the various embodiments of block diagrams for gastric electrical stimulation systems, devices, components and methods, the treatment of nausea and vomiting, and temporary gastric stimulation can be employed in different combinations, permutations and variations.

### 1. Gastric Electrical Stimulation Systems, Devices, Components and Methods

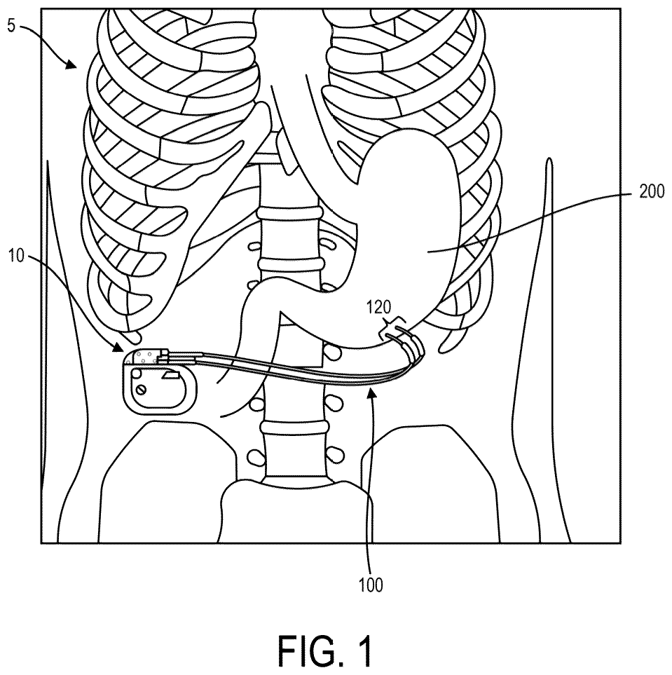

FIGS. 1 through 6 show various embodiments of electrical/electronic block diagrams for implantable pulse generators (IPGs) 10 and/or temporary gastric electrical stimulator (GES) 10, and various components associated therewith, which may also be adapted to, employed in, or correspond to at least portions of temporary gastric electrical stimulator 10 (temporary GES 10).

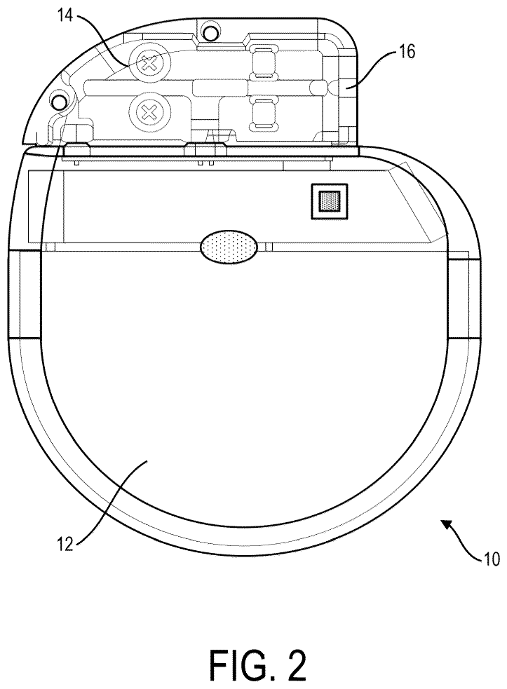

In FIG. 1, there is shown an IPG 10 implanted in stomach 200 of patient 5 with medical electrical leads 100 extending therefrom. In FIG. 2 there is shown one embodiment of an IPG 10 comprising can 12, connector block 14 and lead connection port 16. As used herein, the term “IPG” or “IPG 10” can mean an implantable device, and in some contexts also mean or apply to an external non-implantable device (e.g., an external temporary electrical stimulator or temporary gastric electrical stimulator (GES) 10).

In some embodiments, and as shown in FIGS. 1 and 2, a gastric electrical system (GES) includes an implantable or external pulse generator 10 and leads 100. Electrodes 120 of leads 100 are typically implanted in the wall of stomach 200. Stimulator or IPG 10 is usually implanted in the abdomen, just under the skin.

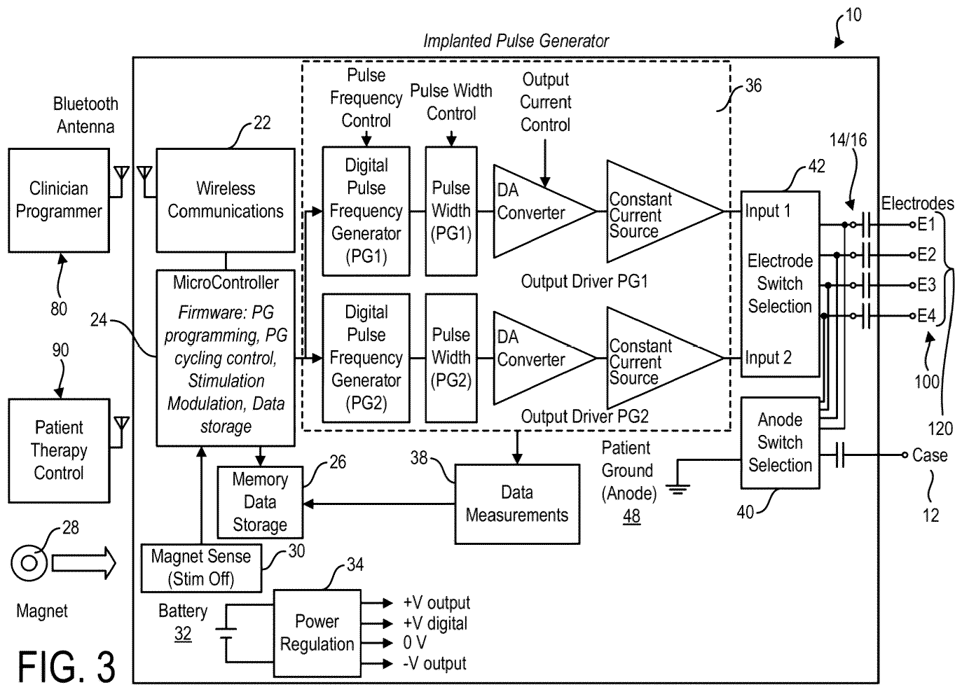

With reference to FIG. 2, in some embodiments there is provided an implantable pulse generator (IPG) 10 comprising electronics and a battery disposed within a hermetically sealed metal enclosure 12. An accelerometer may be included the IPG. A temperature sensor may also be disposed within or outside the IPG, or otherwise be operably connected thereto (e.g., wirelessly from an external device such as a smartwatch). See, for example, the illustrative but non-limiting embodiments shown in FIGS. 3-6.

Referring now to FIGS. 3 through 6, there are shown block diagrams according to various different embodiments of gastric electrical stimulation systems, devices, components and methods. In the various embodiments illustrated in FIGS. 3 through 6, implantable pulse generator (IPG) system 10 comprises 5 principal components: (1) IPG or implantable pulse generator 10; (2) implantable medical electrical stimulation leads 100, which in one embodiment comprise stimulating electrodes 120 or E1 through E4; (3) clinician controller/programmer 80; (4) magnet 28, and (5) patient therapy controller/programmer 90. IPG 10 provides electrical stimulation pulses between electrodes 120 located on lead 100 and/or anodic IPG case 12 (see anode switch selection 40). Clinician controller/programmer 80 permits a health care provider to adjust the operational parameters of IPG 10 and to review data stored in IPG 10 during IPG 10 and lead 100 implantation within patient 5 and/or during follow-up patient visits. An external magnet 28 can be utilized to provide the capability for a clinician or patient to temporarily turn off the IPG for a predetermined period of time (e.g., 24 hours), to turn the IPG back on, or to change some other operational parameter of IPG 10. The patient therapy controller/programmer can be configured to permit patient 5 to receive messages, status information, and other data from IPG 10, and may also be used to make simple adjustments to stimulation parameters as prescribed by the clinician.

Within IPG 10, there is a pulse generator circuit 36 that in one embodiment includes at least two independent pulse generator channels. Each pulse generator channel generates signals having certain stimulation pulse frequency, stimulation pulse width, and stimulation pulse amplitude parameters. Stimulation pulse amplitude generation can be configured to permit selectable stimulation amplitudes in combination with a constant current output source. In one embodiment, and by way of non-limiting example, each stimulation channel can be selected by a multiplexer switching circuit 42 to act as at least one of four cathodic electrodes 120. Note that many other electrode configurations and number of electrodes are contemplated, such as bipolar electrodes, unipolar electrodes, tripolar electrodes, more than 2 electrodes, more than 4 electrodes, employing case 12 as an anode, employing one or more lead electrodes as anodes, and so on. Ground can also be switched between at least four anodic electrodes or the IPG case 12 as the patient ground or on lead 100 as ground electrodes.

In some embodiments, microcontroller 24 provides control to stimulation circuit 36, and together are configured to: (1) generate therapeutic On/Off cycling stimulation therapy signals; (2) provide an interface between wireless communications circuit 22 and adjustment of stimulation circuit 36; (3) provide on-demand and/or real real-time control and/or sensing of selected measurements (e.g., lead impedance, battery voltage, etc.); (4) provide control of therapeutic signal delivery scheduling, and: (5) provide control for programming data and measured data 38 in memory storage 26. In some embodiments, memory included in microcontroller 25 contains firmware executable by microcontroller 25. Note that microcontroller 24 may be any one or more of a CPU, a controller, a microcontroller, a processor, a microprocessor, or any other suitable processing device

In some embodiments, wireless communications circuit 22 may be configured to receive communication signals from clinician controller/programmer 80 and/or a patient controller/programmer 90. Wireless communications circuit 22 can be configured to provide capabilities to adjust parametric operational settings of IPG 10, which settings may also be stored in memory data storage 26 and executed by processor/microcontroller 24 within IPG 10, or by another computing device, for review by the clinician or patient.

As noted above, external magnet 28 and magnet sense circuit 30 may be configured to temporarily turn off or otherwise modify the electrical stimulation regime provided by IPG 10 (e.g., turn stimulation off or on for a predetermined period of time such as 24 hours). In an embodiment where temporary gastric electrical stimulation is to be provided, external magnet 28 and/or patient therapy controller/programmer 90 may also be configured to adjust down or up the amplitude or other stimulation signal parameters of IPG 10 using, by way of non-limiting example, one or more predefined sets of stimulation parameters or levels relating to amplitude, frequency, phase, waveform selection or type, duty cycle, on/off periods, ramping, current levels, etc., as well as to as turn off or on the delivery of electrical stimulation by IPG 10.

In one embodiment, power source 32 is provided by a primary or secondary (rechargeable) battery. In one embodiment, power regulation circuit 34 is configured to provide regulated output voltages to: (1) a digital voltage supply: (2) a positive output voltage supply; (3) a negative output voltage supply; and (4) a circuit ground. The positive and negative output voltage supplies may also be adjusted based on stimulation signal output amplitude demand to optimize power consumption.

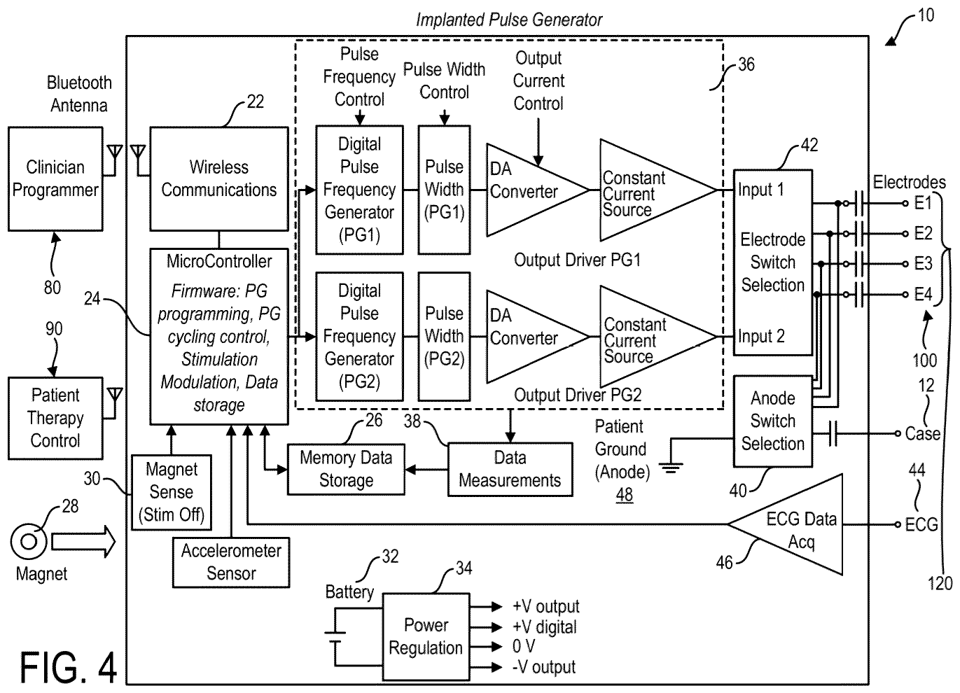

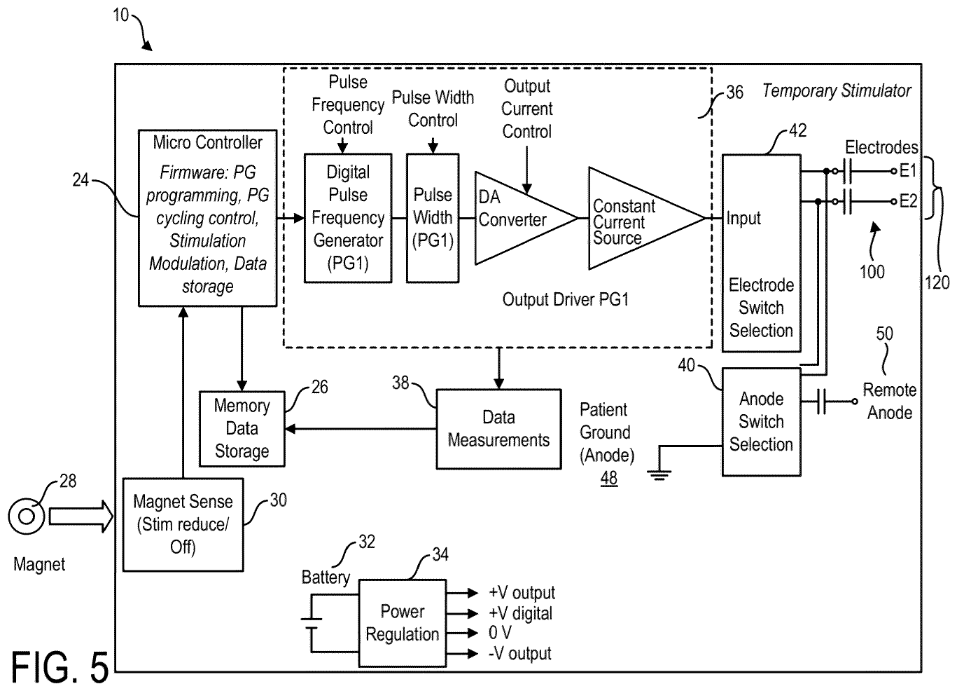

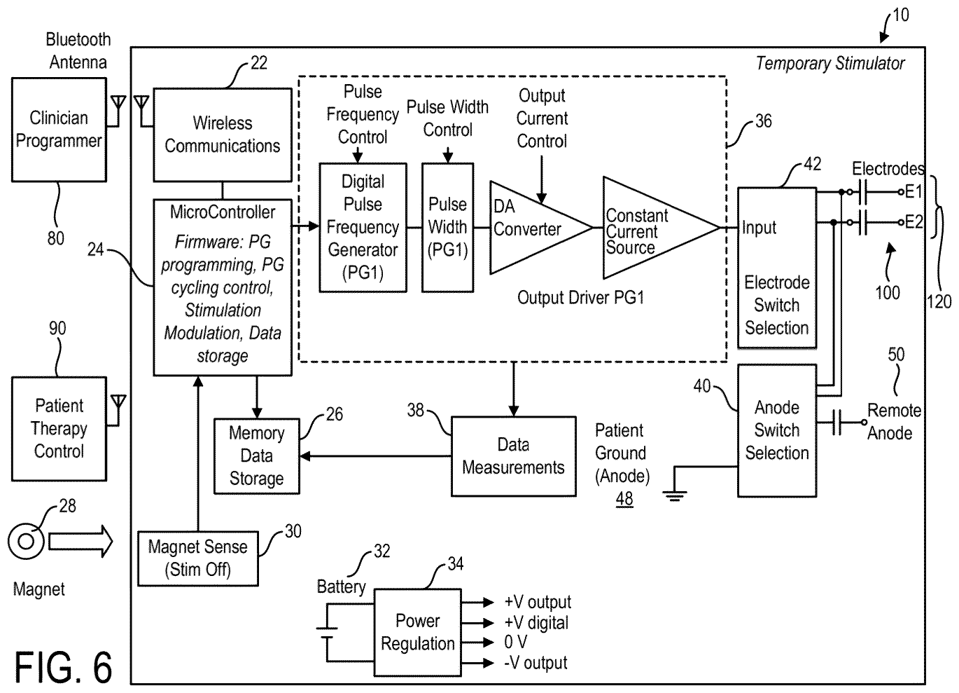

FIGS. 4 through 6 show block diagrams configured for specific purposes and capabilities of gastric electrical stimulation systems, devices, components and methods. The block diagram of FIG. 4 comprises additional sensory inputs for an accelerometer sensor and an electrocardiogram (ECG) signal input 44 to ECG data acquisition circuit 46. The block diagram of FIG. 5 corresponds to one embodiment of a temporary gastric electrical stimulator that does not feature wireless communication capabilities. The block diagram of FIG. 6 corresponds to one embodiment of a temporary gastric electrical stimulator that does feature wireless communication capabilities, which permit electrical stimulation signal adjustment and fine-tuning after implantation using wireless communication.

Continuing to refer to FIGS. 3-6, IPG 10, clinician controller/programmer 80, and/or patient therapy controller/programmer 90 may also be configured to include operable connections to other systems, computers, computing devices, servers, LAN, WANs, the Cloud, and other computing and/pr processing devices through internet connections, WiFi and Bluetooth connections, LAN and WAN connections, and other connecting means, systems and devices known to those skilled in the art of computing systems, devices, and components.

For example, a computer or other computing devices may be configured to receive operator inputs from IPG 10, clinician controller/programmer 80, and/or patient therapy controller/programmer 90. Outputs from such a computer may be displayed on display or monitor or other output devices, and the computer may also be operably connected to a remote computer or analytic database or server. At least one of IPG 10, clinician controller/programmer 80, and/or patient therapy controller/programmer 90 and/or components, devices, modules or systems connected thereto may be operably connected to other components or devices by wireless (e.g., Bluetooth) or wired means. Data may be transferred between components, devices, modules or systems through hardwiring, by wireless means, or by using portable memory devices such as USB memory sticks. Data received or transferred to IPG and/or temporary GES 10 from controllers/programmers 80 and 90, or from or by other external computing and/or communication devices may also be stored, processed and/or analyzed in other computing systems, computers, computing devices, servers, LANs, WANs, the Cloud, and other computing and/pr processing devices through internet connections, WiFi and Bluetooth connections, LAN and WAN connections, and other connecting means, systems and devices known to those skilled in the art of computing systems, devices, and components.

A computing device or computer may also be appropriately configured and programmed to receive or access gastric electrical or EGG signals sensed in stomach 200 of patient 5, and to analyze or process such EGG signals in accordance with the methods, functions and logic disclosed and described herein so as to permit analysis of EGG information. This, in turn, can make it possible to diagnose the gastric disorder or irregularity from which patient 5 suffers.

In view of the structural and functional descriptions provided herein, those skilled in the art will appreciate that portions of the devices and methods described herein may be configured as methods, data processing systems, or computer algorithms. Accordingly, these portions of the devices and methods described herein may take the form of a hardware embodiment, a software embodiment, or an embodiment combining software and hardware. Furthermore, portions of the devices and methods described herein may be a computer algorithm or method stored in a computer-usable storage medium having computer readable program code on the medium. Any suitable computer-readable medium may be utilized including, but not limited to, static and dynamic storage devices, hard disks, optical storage devices, and magnetic storage devices.

Certain embodiments of portions of the devices and methods described herein are also described with reference to block diagrams of methods, systems, and computer algorithm products. It will be understood that such block diagrams, and combinations of blocks diagrams in the Figures, can be implemented using computer-executable instructions. These computer-executable instructions may be provided to one or more processors of a general-purpose computer, a special purpose computer, or any other suitable programmable data processing apparatus (or a combination of devices and circuits) to produce a machine, such that the instructions, which executed via the processor(s), implement the functions specified in the block or blocks of the block diagrams.

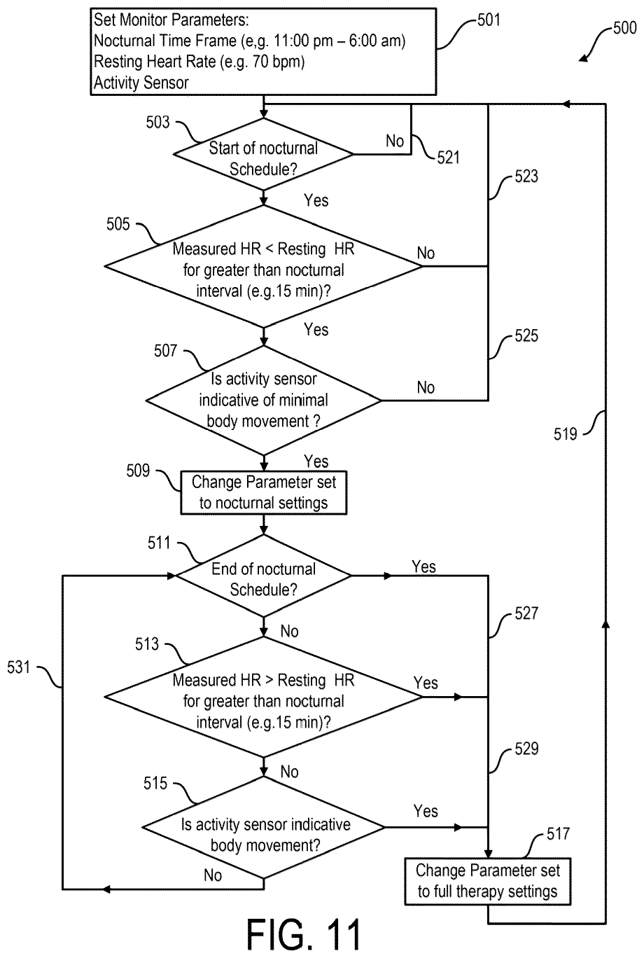

These computer-executable instructions may also be stored in a computer-readable memory that can direct IPG 10, clinician controller/programmer 80, patient therapy controller/programmer 90, and/or a computer or other programmable data processing apparatus to function in a particular manner, such that the instructions stored in the computer-readable memory result in an article of manufacture including instructions which implement the function specified in an individual block, plurality of blocks, or block diagram. The computer program instructions may also be loaded onto IPG 10, clinician controller/programmer 80, patient therapy controller/programmer 90, and/or a computer or other programmable data processing apparatus to cause a series of operational steps to be performed on the computer or other programmable apparatus to produce a computer implemented process such that the instructions which execute on IPG 10, clinician controller/programmer 80, patient therapy controller/programmer 90, and/or a computer or other programmable data processing apparatus provide steps for implementing the functions specified in an individual block, plurality of blocks, or block diagram. See, for example, FIGS. 11, 12, 13, and 14 and portions of the Detailed Description corresponding thereto, where computer code corresponding to methods 500, 600, 700, 800 and other methods/algorithms may be formatted for loading into a memory of processor/microcontroller 24 of IPG 10 and/or temporary GES 10 for execution thereby.

In this regard, FIGS. 3-6 illustrate only certain examples of gastric electrical stimulation signal delivery and sensing systems (which, by way of example, can include multiple computers or computer workstations) that can be employed to execute one or more embodiments of the devices and methods described and disclosed herein, such as devices and methods configured to acquire and process sensor or electrode data, to process image data, and/or transform sensor or electrode data and image data associated with the analysis of gastric electrical activity and the carrying out of the combined gastric electrical mapping and analysis of the patient's stomach 200 and gastric electrical stimulation therapy delivered thereto.

The various computing devices described and disclosed herein, including IPG 10, temporary GES 10, clinician controller/programmer 80, patient therapy controller/programmer 90, and/or a computer or other programmable data processing apparatus, can be implemented on, or operably connected to, one or more general purpose or specialized computer systems or networked computer systems, embedded computer systems, routers, switches, server devices, client devices, various intermediate devices/nodes, and/or standalone computer systems.

In one embodiment, IPG 10 or temporary GES 10 includes processing unit 24 (which may comprise a CPU, controller, microcontroller, processor, microprocessor or any other suitable processing device), system memory 26, and in some embodiments a system bus that operably connects various system components, including system memory data storage 26, to processing unit 24. Multiple processors and other multi-processor architectures also can be used to form processing unit 24. In some embodiments, the system bus can comprise any of several types of suitable bus architectures, including a memory bus or memory controller, a peripheral bus, and/or a local bus. System memory data storage 26 can include read only memory (ROM) and/or random-access memory (RAM), as can memory in processing unit or controller 24. A basic input/output system (BIOS) can be stored in the ROM and contain basic routines configured to transfer information and/or data among the various elements within IPG 10 or temporary GES 10.

### 2. Treatment of Nausea and Vomiting

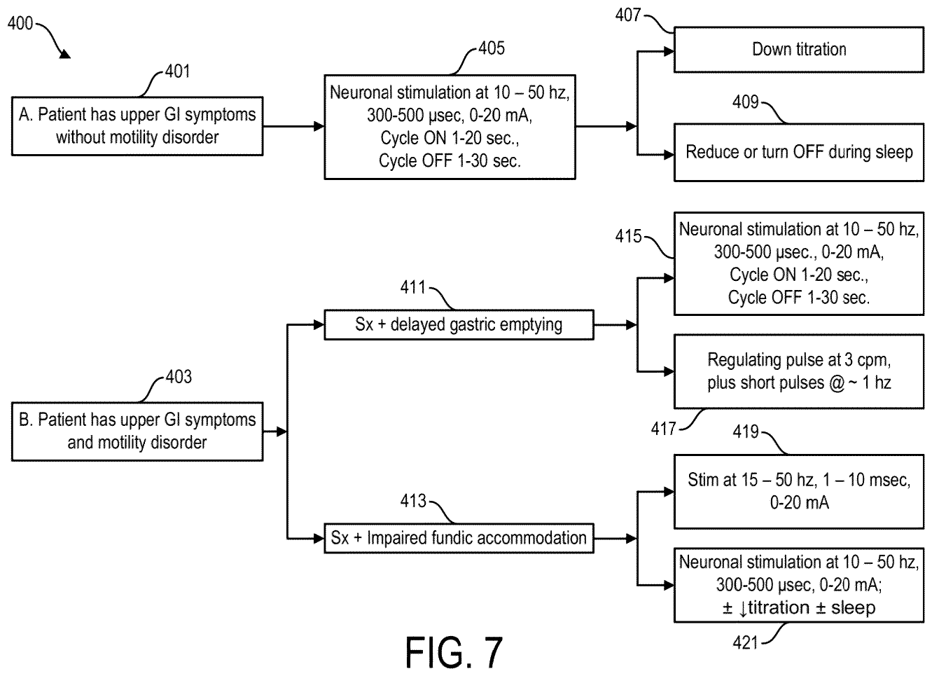

Patients may have severe or moderate gastrointestinal (GI) symptoms alone or accompanied by different dysmotility disorders. We describe various embodiments of methods, systems, devices and components to treat these two different symptom conditions with gastric electrical stimulation. See, for example, FIG. 7, where method 400 illustrates aspects of some embodiments described and disclosed in further detail below.

In FIG. 7, at step 401 patients with GI symptoms but without motility disorders are treated using subsequent steps 405, 407 and/or 409. Conversely, at step 403 patients with GI symptoms and with motility disorders are treated using subsequent steps 411, 415 and/or 471, or steps 413, 419 and/or 421.

Patients suffering from the foregoing symptoms sometimes require relatively high stimulation parameter levels, which can result in abnormally high power consumption of IPG 10, substantially shortening battery longevity. Currently, most clinicians initially program a gastric electrical stimulator (GES) implanted in a patient to employ relatively conservative or low stimulation parameters, and gradually increase stimulation levels over time. This can require significant time on the part of both the physician and patient to establish settings for effective therapy.

In FIG. 7, at step 401 patients with GI symptoms but without motility disorders are treated using subsequent steps 405, 407 and/or 409. In 405, a patient is treated neuronal gastric electrical stimulation using stimulation parameter in the range of a) pulse frequency 10-50 hz, b) pulse width 300-500 μsec, and c) amplitude 0-20 mA, and d) cycle ON in the range of 0.1-20 sec, and cycle OFF in the range of 1-30 sec. Using such stimulation parameters, patients may further be treated in two ways as in steps 407 and 409, either separately or sequentially. Step 407 represents a method of down-titrating stimulation parameters to shorten the time required on the part of both physician and patient to establish settings for effective therapy. In step 407, the initial stimulation parameters may be set to high values falling within the capability of the stimulation device, for example 20 mA, 28 hz, 330 μsec, and cycling 3 sec on/2 sec off. If the patient is responsive to stimulation after a period of 2 to 30 days, stimulation parameters may be reduced in a stepwise manner at intervals of, by way of non-limiting example, 2 to 30 days wherein amplitude, frequency and cycling ON/OFF times down titrated (i.e., reduced) until the patient indicates that the initial response to stimulation is diminished.

Step 409 represents a nocturnal cycling protocol to reduce overall energy consumption of the therapy. In step 409, stimulation parameters, including amplitude, frequency, pulse width, and ON/OFF cycling, may be significantly reduced, or the device may be turned off, during sleep. Upon waking, the stimulator may be turned on, or the parameters reset to the parameters found to be effective in step 407. The process to reducing stimulation parameters during sleep or turning the device off and then back on, may be accomplished by a timer set according to the patient's particular sleep habits. Alternatively, stimulation parameters may be reduced or the device turned OFF and then back ON by using a suitable sensor for detecting sleep including sensors for activity, heart rate, or respiration.

Patients are known to experience moderate to severe gastro-intestinal (GI) symptoms in association with motility disorders. Such symptoms may include nausea, early satiety, postprandial fullness, abdominal pain. Notably, some patients experience similar symptoms, but without having gastric motility disorders. Gastric electrical stimulation (GES) has been shown to be effective in treating such symptoms using neurostimulation parameters within the capability of the existing Enterra gastric electric stimulation system (e.g., pulse width up to 450 μsec. frequencies in the range of 10-110 Hz, and amplitudes up to 20 mA).

A. GES Treatment of Symptoms in Patients with GI Motility Disorders

Referring now to FIG. 7 and steps 401-409, In patients with motility disorders and severe GI symptoms such as nausea, early satiety, postprandial fullness, and abdominal pain, GES methods have been implemented using the Enterra gastric electric stimulation system (e.g., pulse width up to 450 μsec, frequencies in the range of 10-50 Hz or 10-110 Hz, and amplitudes up to 20 mA). Such a therapeutic approach may be used with or without the programming protocols or nocturnal cycling protocols described herein.

### B. Method for GES Treatment of Delayed Gastric Emptying

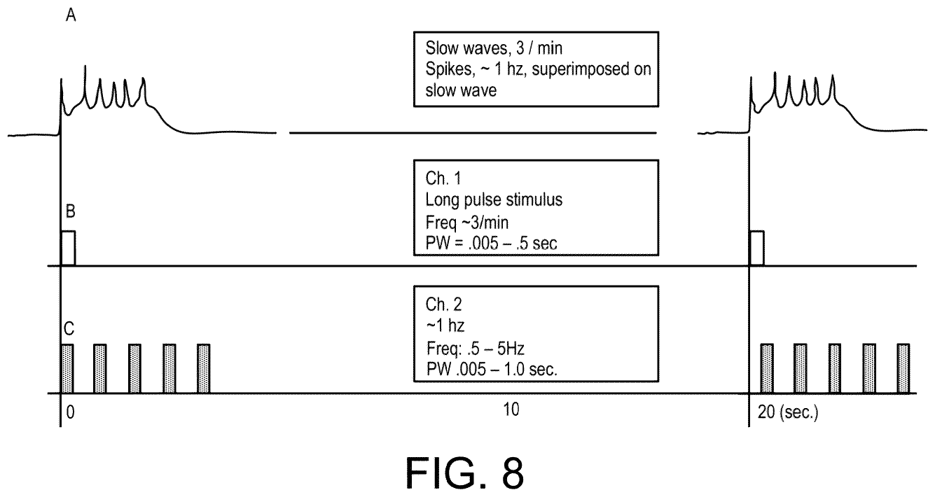

Delayed gastric emptying is typically associated with one or more of nausea, vomiting, early satiety, postprandial fullness, and abdominal pain. Treatment of delayed gastric emptying using GES methods can improve these symptoms, or improve nutritional status, in patients receiving GES therapy. In one embodiment, electrical stimuli are applied to the stomach (using one or more pairs of indwelling electrodes connected to a pulse generator) in such a manner as to mimic the combination of the gastric slow wave and gastric spike activity. See, for example, FIG. 8.

In FIG. 8, Tracing A is a recording from a 1978 study done by Szurszewski et al. (1978), “A study of the canine gastric action potential in the presence of tetraethylammonium chloride.” The Journal of Physiology, 277(1), 91-102 (which publication is hereby incorporated by reference into the specification of the present patent application, in its entirety), and shows a gastric slow wave with “spike” signals super-imposed on the slow wave. The spike signals indicate smooth muscle depolarization, i.e., contraction. Tracing B shows a stimulus which can be applied on a first channel to the stomach at the native slow wave frequency (e.g., 3 waves/minute) to regulate gastric smooth muscle activity. Note that in patients with gastroparesis, the slow wave frequency may be too fast, slow, or may be irregular or disordered. Tracing C shows a burst of pulses which may be applied on a second channel to the stomach so as to mimic spike activity in healthy subjects. This stimulus occurs at approximately 1 Hz, or between about 0.5 Hz and about 5 Hz, and may be initiated at the beginning or just after regulating pulse in Tracing B. The stimulus shown in Tracing C may be controlled so that it occurs shortly after the initiation of a meal. This may be accomplished by means of a timer, a patient controller, or by means of a sensor that detects the onset of a meal and communicates that event to the implantable stimulator. The stimulus of Tracing C may be terminated after a programmable period of time, say after 1 to 4 hours.

### C. Method for GES Treatment of Fundic Accommodation Disorders.

A range of GI symptoms including nausea, early satiety, postprandial fullness, abdominal pain are often associated with impaired fundic accommodation.

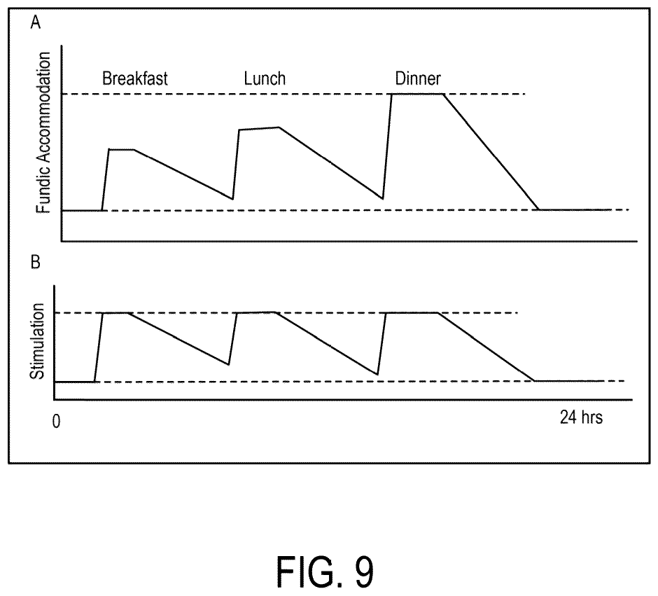

Tracing A in FIG. 9 illustrates a fundic accommodation response in normal subjects' meals during a circadian 4-hour period. The fundus begins to relax so that the volume of a meal can be accommodated without an increase in intragastric pressure. Larger meals may result in a larger accommodation response than smaller meals. After the meal has been completed, the fundus tonic contraction resumes, and accommodation decreases over time.

Patients with impaired fundic accommodation do not have the normal accommodation, as described above. To improve subjects with impaired fundic accommodation, a stimulus can be applied over a 24-hour period, as illustrated in Tracing B in FIG. 9. Here the stimulus is applied to mimic the time course of the desired accommodation response over a 24-hour circadian cycle.

In such a method, electrical stimuli are applied to the stomach with indwelling electrodes connected to a pulse generator mor IPG 10. In one embodiment, the electrical stimulus signals may have a pulse width of 0.1 to 10 msec, and a frequency of 10-110 Hz. The electrical stimulation pulses may be delivered continuously according to Tracing B in FIG. 9, or may be delivered in bursts of pulses, where burst length and inter-burst period may be adjusted by means of a controller or physician programmer. The electrical stimulus signals may be initiated at the onset of a meal, or controlled by a timer, a patient controller/programmer 90, or a sensor capable of detecting the onset of a meal. The stimulus may be terminated when the meal has ended and may be controlled by a timer, a patient controller, or sensor capable of detecting the completion of a meal.

### D. GES Programming Protocols Employing Successive Down-Titration of Programming Parameter Levels

One problem when applying GES parameters for GI symptoms is difficulty in rapidly or efficiently determining how to arrive at effective stimulation parameters for each patient. Currently, most clinicians program the electrical stimulation generator (which may be a temporary external or implantable electrical stimulator) or implantable pulse generator (IPG 10) implanted in the patient with relatively conservative or low electrical stimulation parameters, and then gradually increase stimulation parameter levels over time. Such an approach often requires significant time on the part of both the physician and patient. As described and disclosed herein, an alternative approach is to start out using high level electrical stimulation parameters as part of a programming protocol, as there appear to be few if any adverse events related to the use of high level stimulation parameters. In one embodiment, the programming protocol starts with high level stimulation parameters and then down-titrates stimulation parameter levels until efficacy is determined to have diminished.

In embodiments relating to treatment of one or more gastrointestinal disorders of the patient such as one or more of nausea, vomiting, early satiety, postprandial fullness, and abdominal pain, methods, systems, devices and components are provided that result in more rapid and accurate programming and determination of gastric stimulation parameters for the patient compared to conventional gastric stimulation programming techniques or methods, and which can also reduce power requirements and battery drain.

In some such embodiments, there is provided a method of electrically stimulating a portion of a patient's stomach to treat the foregoing one or more gastrointestinal disorders of the patient. In some embodiments, the method comprises implanting an implantable pulse generator (IPG 10) in or near the stomach of the patient and then over a first predetermined period of time, generating first electrical stimulation signals in IPG 10, the first electrical stimulation signals having one or more frequencies ranging between about 2 Hz and about 110 Hz or about 120 Hz, one or more pulse widths ranging between about 100 μsec. and about 10 msec., one or more amplitudes ranging between about 0.1 mA and about 20 mA. This is followed by providing, over at least a portion of the first predetermined period of time, the first electrical stimulation signals through at least one medical electrical lead 100 and one or more electrodes 120 thereof to a portion of stomach 200 of patient 5. Over a second predetermined period of time, second electrical stimulation signals are generated in IPG 10, the second electrical stimulation signals having one or more of frequencies, pulse widths and amplitudes less than one or more of the corresponding frequencies, pulse widths and amplitudes of the first electrical stimulation signals. Over at least a portion of the second predetermined period of time, the second electrical stimulation signals are provided through the at least one medical electrical lead 100 and the one or more electrodes 120 thereof to the portion of stomach 200 of patient 5.

As one or more of the frequencies, pulse widths, and amplitudes of the electrical stimulation signals provided to patient 5 continue to be successively reduced, one or more of the frequency, pulse width, and amplitude parameters of the electrical stimulation signal wherein efficacy in treating the one or more gastrointestinal disorders of the patient is reduced or lost is determined. On the basis of the frequency, pulse width, and amplitude parameters determined to be associated with reduced or lost efficacy in treating the one or more gastrointestinal disorders of the patient, electrical stimulation signals are generated and provided to patient 5 as chronic electrical stimulation signals having one or more of increased frequency, pulse width, and amplitude parameters compared to the frequency, pulse width, and amplitude parameters determined to be associated with substantially reduced or lost efficacy in treating the one or more gastrointestinal disorders of patient 5.

### E. Nocturnal and Circadian Cycling Protocols

Other means for reducing power consumption in a pulse generator or IPG 10 or temporary GES 10 in the application of GES for GI Symptoms are nocturnal and/or circadian cycling protocols. In some embodiments, stimulation parameter levels (such as pulse width, pulse amplitude, duty cycle, frequency, etc.) are reduced during sleep so as to reduce pulse generator power consumption during sleep or rest. Alternatively, output from the pulse generator may be turned off completely during sleep or rest to conserve additional power from the pulse generator. Control of the pulse generator (e.g., IPG 10 or external stimulator or temporary GES 10) may also be accomplished using a sleep detector coupled to the pulse generator to automatically change stimulation parameter levels at the onset of sleep, rest, and/or upon waking.

When programming a pulse generator for a nocturnal cycle, IPG 10 or temporary GES 10 can be nominally programmed to provide a full therapy parameter set during a patient's waking hours (e.g., 7:00 am to 11:00 pm). During sleeping hours (e.g., 11:00 μm to 7:00 am) a secondary parameter set (e.g., reduce pulse frequency and/or reduce percent Cycle On time) could be programmed. The pulse generator 10 can also be configured to monitor sensory data indicative of a patient's sleep state. This feedback can be used to automatically adjust the nocturnal schedule to coincide with the patient's actual sleep period. For example, sensory feedback may include an accelerometer for the detection of body motion.

In some embodiments, when a lower and steady heart rate is detected during a programmed nocturnal schedule, IPG 10 or temporary GES 10 may interpret such a condition as confirmation that the patient is in a sleep state. An accelerometer incorporated into pulse 10 generator may also be used to confirm that that patient 5 is in a non-moving or supine condition.

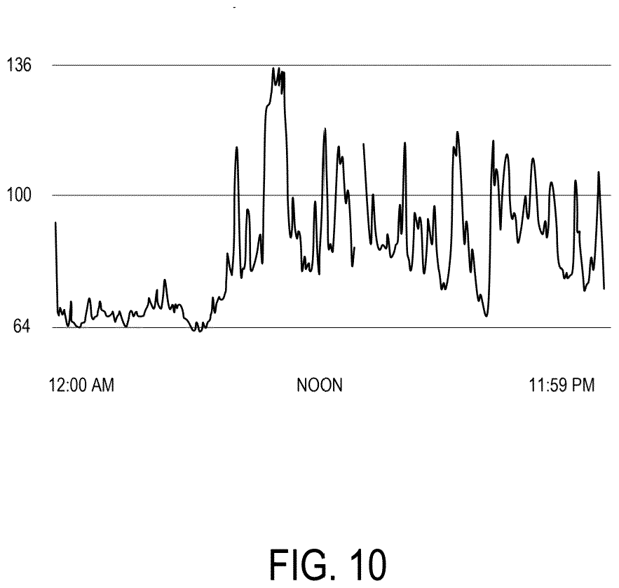

Another example of sensory feedback can be the patient's heart rate. The heart rate can be obtained with an ECG sensor included in pulse generator 10 or one of its leads 100, or in a smart watch or other external device worn by the patient capable of communicating with the pulse generator 10 (e.g., wirelessly or via Bluetooth). This may include providing a recording electrode on the lead body with respect to a ground reference (e.g., pulse generator case 12). Using heart rate and activity sensing is used by many wearable monitoring devices. FIG. 10 shows exemplary heart rate data of a patient over a 24 hour time period. As shown in the example of FIG. 10, heart rate in the patient decreases between midnight and around 6:00 am when the patient is sleeping.

Continuing to refer to FIG. 10, when programming a Gastric Electrical Stimulator (GES) 10 for the nocturnal cycle, the device can be nominally programmed to provide a full therapy parameter set during a patient's waking hours (e.g., 7:00 am to 11:00 pm). During sleeping hours (e.g., 11:00 μm to 7:00 am) a secondary parameter set (e.g., reduce pulse frequency and/or reduce percent cycle on time) could be programmed. The IPG or temporary GES 10 can also monitor sensory data indicative of a patient's sleep state. This feedback can be used to automatically adjust the nocturnal schedule to coincide with the patient's actual sleep period. Note that heart rate and activity sensing can also be accomplished using wearable monitoring devices.

The flow chart shown in FIG. 11 shows one embodiment of a method/algorithm 500 configured to allow IPG 10 or temporary GES 10 to provide greater specificity to automatically adjust electrical stimulation parameters during a nocturnal cycle. Method/algorithm 500 illustrated in FIG. 11 is intended to reduce the stimulation during the daily cycle when electrical stimulation therapy may not be required as much (e.g., during sleeping hours of 11:00 μm to 6:00 am). One method of detecting the patient's sleep condition can be by monitoring the patient's resting heart rate and body motion with an activity sensor. At the beginning of high-level control, it is determined whether the nocturnal schedule time has been initiated. If the time is during the predetermined schedule, controller 24 determines whether a measured heart rate is less than a pre-determined resting heart rate (e.g., 70 bpm) for an interval that may be greater than a predetermined nocturnal interval (e.g., 15 minutes). If the heart rate is less than the resting heart rate, controller 24 checks the activity sensor to determine whether there is minimal body motion. If any of these three conditions are not met, or in some embodiments two of the tree conditions are met, then stimulator 10 remains in full stimulation therapy mode. If all three conditions are met, and in some embodiments two of the three conditions are met, then controller 24 can be configured to change the stimulation operational parameters to a predetermined nocturnal parameter set. With provided electrical stimulation operating within a nocturnal mode, controller 24 monitors the three conditions (end of schedule, heart rate, and activity), or two of such conditions, to determine whether any one of them changes: (1) Has the nocturnal therapy ended its scheduled duration? (2) Is the measured heart rate greater than the resting heart rate for a specified interval?, or (3) Does the activity sensor detect body motion? If any of those conditions are met, then controller 24 changes the operational stimulation parameters back to the full therapy settings.

Continuing to refer to FIG. 11, one embodiment of method/algorithm 500 for providing greater specificity to automatically adjust electrical stimulation parameters during a nocturnal cycle, not intended to be limiting, is illustrated. At step 501, monitoring parameters are set. For example, a nocturnal time frame, resting heart rate threshold, and activity sensor parameters can be set at step 501. At step 503, it is determined whether the nocturnal schedule for patient 5 has begun. If so, at step 505 the resting heart rate of patient 5 is measured. If the measured heart rate of patient 5 is greater than the resting heart rate for the nocturnal interval, step 507 is proceeded to, where an activity sensor in IPG 10 or temporary GES 10 is monitored for minimal body movement. If body movement of patient 5 is minimal, then at step 509 stimulation and optionally other operating parameters of IPG 10 or temporary GES 10 are changed to a set of nocturnal operating parameters. For the remainder of the nocturnal time frame, patient 5 is monitored by IPG 10 or temporary GES 10 at step 511, and if the end of the nocturnal schedule is detected (e.g., by patient activity or movement, or an increase in measured heart rate that exceeds a predetermined resting heart rate threshold), operating parameters of IPG 10 or temporary GES 10 are changed at step 517 to full therapy settings (e.g., daytime schedule). Otherwise, resting heart rate and the activity sensor continue to be monitored at steps 513 and 515.

According to one example of pseudocode not intended to be limiting, one embodiment of method/algorithm 500 for providing greater specificity to automatically adjust electrical stimulation parameters during a nocturnal cycle is shown in Table 1 below. According to the pseudocode of Table 1, a basic framework is provided for a Gastric Electrical Stimulator (GES) 10 to adjust its therapy parameters based on the patient's daily schedule and real-time sensory data to confirm sleep states. Computer code corresponding to the pseudocode shown below for method 500 may be formatted and compiled for loading into data storage memory 26 and execution by processor/microcontroller 24 of IPG 10 and/or temporary GES 10, or for execution by an external computing device.

TABLE 1
One Embodiment of Pseudocode for Method/Algorithm 500

```
// Define the main function
function programGESDevice( ) {
  // Define therapy parameter sets (this is an example)
  daytimeParameters = { startTime: ″07:00″, endTime: ″23:00″, pulseFrequency; high,
cycleOnTime: high }
  nocturnalParameters = { startTime: ″23:00″, endTime: ″07:00″, pulseFrequency: low,
cycleOnTime: low }
  // Set initial parameters to daytime
  currentParameters = daytimeParameters
  // Monitor the patient continuously
  while (true) {
    currentTime = getCurrentTime( )
    // Check if current time falls within the nocturnal schedule
    if (isTimeWithinNocturnalSchedule(currentTime, nocturnalParameters)) {
      // If in nocturnal time, set parameters to nocturnal
      currentParameters = nocturnalParameters
      // Monitor sensory data for sleep state confirmation
      if (isPatientinSleepState( )) {
        applyTherapyParameters(nocturnalParameters)
      }
    } else {
      // If in daytime, set parameters to daytime
      currentParameters = daytimeParameters
      applyTherapyParameters(daytimeParameters)
    }
    // Wait for a short period before checking again. The 1 minute wait time is an
assumption and may get updated.
    sleep(1 minute)
  }
}
// Helper function to check if the current time is within the nocturnal schedule
function isTimeWithinNocturnalSchedule(currentTime, parameters) {
  return currentTime >= parameters.startTime | | currentTime < parameters.endTime
}
// Helper function to determine if the patient is in a sleep state
function isPatientInSleepState( ) {
  heartRate = getHeartRate( )
  motionData = getMotionData( )
  // Check for lower and steady heart rate and non-moving condition
  if (isHeartRateSteadyAndLow(heartRate) && isMotionDataNonMoving(motionData)) {
    return true
  }
  return false
}
// Helper function to check if heart rate is steady and low
function isHeartRateSteadyAndLow(heartRate) {
  steadyThreshold = 5 // Example threshold value for steady heart rate
  lowThreshold = 60 // Example threshold value for low heart rate
  return (getHeartRateVariability(heartRate) < steadyThreshold) && (heartRate <
lowThreshold)
}
// Helper function to check if motion data Indicates non-moving condition
function isMotionDataNonMoving(motionData) {
  return motionData.isNonMoving && motionData.isSupine
}
// Placeholder function to apply therapy parameters to the device
function applyTherapyParameters(parameters) {
  // Code to apply therapy parameters to the GES device
}
// Placeholder function to get the current time
function getCurrentTime( ) {
  // Code to get the current time
}
// Placeholder function to get the heart rate
function getHeartRate( ) {
  // Code to get the heart rate from ECG sensor
}
// Placeholder function to get heart rate variability
function getHeartRateVariability(heartRate) {
  // Code to calculate heart rate variability
}
// Placeholder function to get motion data from accelerometer
function getMotionData( ) {
  // Code to get motion data from accelerometer
}
// Placeholder function to pause execution for a given duration
function sleep(duration) {
  // Code to pause execution
}
```

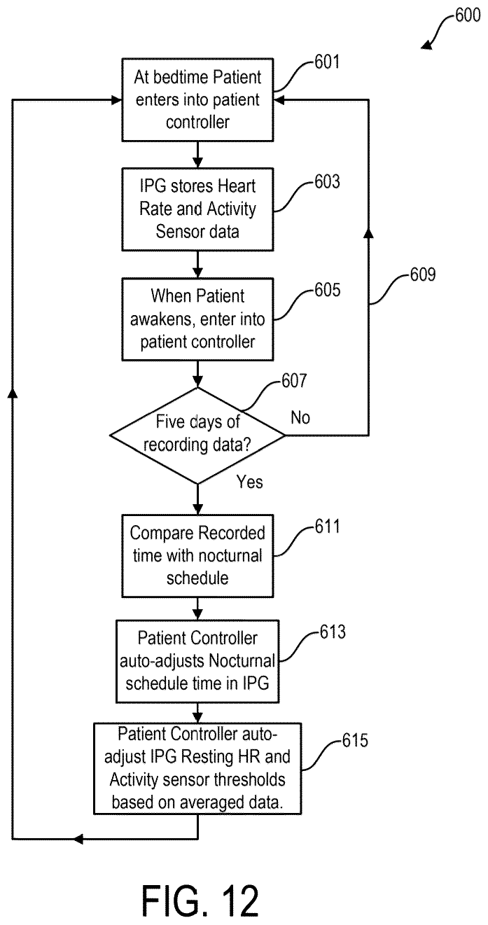

In another embodiment, and with reference to FIG. 12, there is provided a method/algorithm 600 configured to provide greater specificity to adapting the adjustment of the nocturnal schedule to a patient's circadian schedule, which can include providing patient 5 with an external instrument device such as a smart watch to assist in the process of learning or determining when the patient is in a sleep state. Such an external device can be configured to permit the patient to enter a time mark when they are going to sleep. This event can be received by the pulse generator or other device to record heart rate and/or activity sensor data at timed intervals until the patient wakes up. Having such data permits the pulse generator or other device to automatically adjust the thresholds for the resting (i.e., lower) heart rate during sleep and the activity as sensed by the accelerometer representative of the patient's sleeping condition. The flow chart in FIG. 12 illustrates one such embodiment and method/algorithm 600.

Continuing to refer to method/algorithm 600 of FIG. 12, and in one embodiment, patient 5 is provided with an external controller/programmer device 90 which is configured to provide wireless communications to implanted GES device or IPG 10. When patient 5 is ready to go to bed, they can activate the external controller/programmer 90 to inform IPG 10, which will then establish a baseline for monitoring the patient's heart rate and the activity sensor during the nocturnal time period. When the patient awakens, they once again they activate the external controller/programmer 90 to mark the end of the sleep cycle. In one embodiment, after five days of recording such data, the patient controller/programmer 90 adjusts the nocturnal schedule, resting heart rate and activity sensor thresholds to new settings based on averages obtained from the already-recorded data. Auto-adjustment of these settings can continue beyond the initial 5 day period and adapt to changes in the patient's circadian sleep patterns.

Continuing to refer to method/algorithm 600 of FIG. 12, at step 601, at bedtime patient 5 accesses patient controller/programmer 90 to indicate that patient 5 is going to sleep, which activates IPG or temporary GES 10 at step 603 to continuously store patient 5's current heart rate sensor and activity sensor data while patient 5 is sleeping. At step 605, when the patient awakes from sleep, this information is also entered into controller/programmer 90 by patient 5. At step 607, the foregoing data are recorded, by way of non-limiting example, for five successive days. If not, steps 601 through 607 are repeated. If so, at step 611 the time which patient 5 went to sleep over the 5 day period is compared to the predetermined nocturnal schedule for patient 5. At step 613, patient controller/programmer 90 updates or auto-adjust the predetermined nocturnal schedule in accordance with the results of step 611. At step 615, patient controller/programmer 90 updates or auto-adjusts resting heart rate sensor and activity sensor thresholds based on averaged data that have been obtained and analyzed for the 5 day period. The entire process is then repeated, beginning with step 601.

According to one example of pseudocode not intended to be limiting, one embodiment of method/algorithm 600 configured to provide greater specificity greater specificity to adapting the adjustment of a nocturnal schedule to a patient's circadian schedule is shown in Table 2 below. According to the pseudocode of Table 2, a basic framework is provided for high-level control to adjust GES 10 based on a nocturnal schedule and a method for adapting the schedule based on patient input, as shown in FIG. 12. The latter part of the code handles the process of learning and adjusting thresholds based on data collected while the patient is asleep. Computer code corresponding to the pseudocode shown below for method 600 may be formatted and compiled for loading into data storage memory 26 and execution by processor/microcontroller 24 of IPG 10 and/or temporary GES 10, or for execution by an external computing device.

TABLE 2
One Embodiment of Pseudocode for Method/Algorithm 600

```
// Define the main function
function controlGESDevice( ) {
  // Define therapy parameter sets
  daytimeParameters = { pulseFrequency: ″high″, cycleOnTime: ″high″ }
  nocturnalParameters = { pulseFrequency: ″low″, cycleOnTime: ″low″ }
  // Define constants. This is an example
  nocturnalStartTime = ″23:00″
  nocturnalEndTime = ″06:00″
  restingHeartRateThreshold = 70 // bpm
  nocturnalInterval = 15 // minutes
  // Set Initial parameters to daytime
  currentParameters = daytimeParameters
  // Initialize array to store recorded data
  recordedData = [ ]
  // Monitor the patient continuously
  while (true) {
    currentTime = getCurrentTime( )
    if (isTimeWithinNocturnalSchedule(currentTime, nocturnalStartTime,
nocturnalEndTime)) {
      // Check If heart rate is less than the resting heart rate threshold for the specified
interval
      if (isHeartRateBelowThresholdForInterval(restingHeartRateThreshold,
nocturnalInterval)) {
        // Check if activity sensor indicates minimal body motion
        if (isBodyMotion Minimal( ) {
          // All conditions are met, switch to nocturnal parameters
          applyTherapyParameters(nocturnalParameters)
        } else {
          // Body motion detected, stay in full stimulation therapy mode
          applyTherapyParameters(daytimeParameters)
        }
      } else {
        // Heart rate not below threshold, stay in full stimulation therapy mode
        applyTherapyParameters(daytimeParameters)
      }
    } else {
      // Not within nocturnal time, use daytime parameters
      applyTherapyParameters(daytimeParameters)
    }
    // Monitor conditions to revert from nocturnal mode
    If (isOperatingInNocturnalMode(currentParameters) {
      If (isTimeOutsideNocturnalSchedule(currentTime, nocturnalStartTime,
nocturnalEndTime) | |
        isHeartRateAboveThreshold(restingHeartRateThreshold, nocturnalInterval) | |
        isBodyMotionDetected( )) {
        // Revert to full therapy settings
        applyTherapyParameters(daytimeParameters)
      }
    }
    // Check if it's time to mark the end of the sleep cycle
    If (IsTimeToMarkEndOfSleepCycle( )) {
      // Mark end of sleep cycle and adapt nocturnal schedule based on recorded data
      endOfSleepCycle( )
      adaptNocturnalScheduleBasedOnRecordedData(recordedData)
      // Clear recorded data for next cycle
      recordedData = [ ]
    }
    // Wait for a short period before checking again. The 1 minute interval is an assumption
and may get updated.
    sleep(1 minute)
  }
}
// Helper function to check if it's time to mark the end of the sleep cycle
function isTimeToMarkEndOfSleepCycle( ) {
  // Code to check if it's time to mark the end of the sleep cycle
}
// Helper function to mark end of sleep cycle and record data
function endOfSleepCycle( ) {
  // Code to mark end of sleep cycle
  // Record heart rate and activity sensor data at timed intervals until the patient wakes up
  // Store recorded data in the recordedData array
  // For demonstration purposes, let's assume we have recorded heart rate and activity
sensor data
  // Here, we're just simulating the data for the sake of completion
  recordedData = [
    { heartRate: 60, activity: 0.2 },
    { heartRate: 62, activity: 0,25 },
    { heartRate: 65, activity: 0.3 },
    // More simulated data points
  ]
}
// Function to adapt the nocturnal schedule based on recorded data
function adaptNocturnalScheduleBasedOnRecordedData(recordedData) {
  // Check if recorded data is null or empty
  if (recordedData.length === 0) {
    // Handle the scenario when there is no recorded data
    // For example, set thresholds to default values or maintain previous values
    setDefaultThresholds( )
    return
  }
  // Calculate 5-day average for heart rate and activity sensor data
  heartRateSum = 0
  activitySum = 0
  for (dataPoint in recordedData) {
    heartRateSum += dataPoint.heartRate
    activitySum += dataPoint.activity
  }
  avgHeartRate = heartRateSum / recordedData.length
  avgActivity = activitySum / recordedData.length
  // Adjust thresholds based on the average recorded data
  restingHeartRateThreshold = avgHeartRate
  activityThreshold = avgActivity
}
// Helper function to set default thresholds or maintain previous values
function setDefaultThresholds( ) {
  // Code to set default thresholds or maintain previous values
}
// Helper function to check if the current time is within the nocturnal schedule
function isTimeWithinNocturnalSchedule(currentTime, startTime, endTime) {
  return currentTime >= startTime | | currentTime < endTime
}
// Helper function to check if the current time is outside the nocturnal schedule
function isTimeOutsideNocturnalSchedule(currentTime, startTime, endTime) {
  return currentTime < startTime && currentTime >= endTime
}
// Helper function to determine if heart rate is below threshold for a specified interval
function isHeartRateBelowThresholdForInterval(threshold, interval) {
  // Code to check if heart rate is below threshold for a specified interval
}
// Helper function to check if body motion is minimal
function isBodyMotionMinimal( ) {
  // Code to check if body motion is minimal
}
// Helper function to determine if heart rate is above threshold for a specified interval
function isHeartRateAboveThreshold(threshold, interval) {
  // Code to check if heart rate is above threshold for a specified interval
}
// Helper function to check if body motion is detected
function isBodyMotionDetected( ) {
  // Code to check if body motion is detected
}
// Placeholder function to apply therapy parameters to the device
function applyTherapyParameters(parameters) {
  // Code to apply therapy parameters to the GES device
}
// Placeholder function to get the current time
function getCurrentTime( ) {
  // Code to get the current time
}
// Placeholder function to get the heart rate
function getHeartRate( ) {
  // Code to get the heart rate from ECG sensor
}
// Placeholder function to get motion data from accelerometer
function getMotionData( ) {
  // Code to get motion data from accelerometer
}
// Placeholder function to pause execution for a given duration
function sleep(duration) {
  // Code to pause execution
}
```

### F. Sensing and Detecting Gastric Disorders

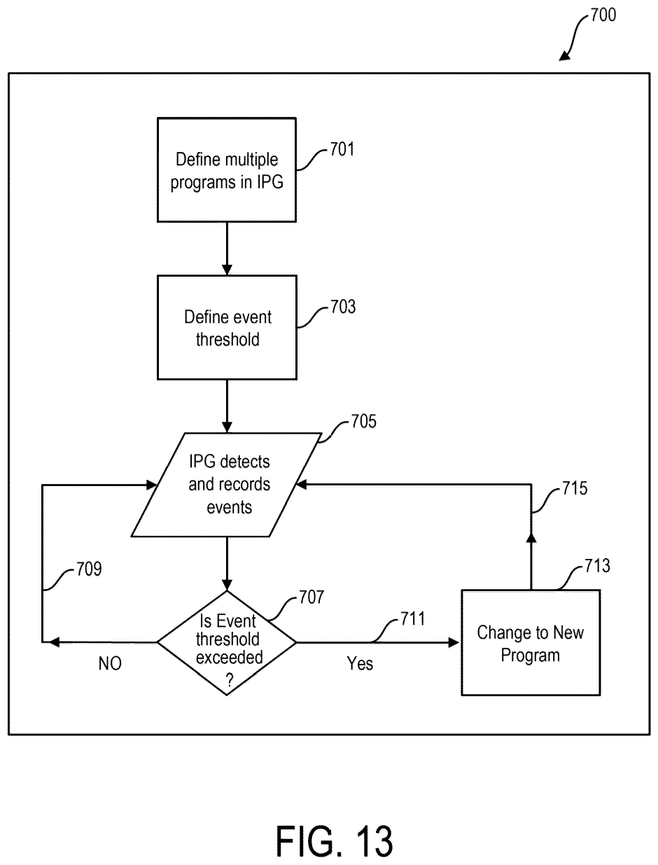

Referring now to FIG. 13, there is illustrated one embodiment of a method/algorithm 700 for IPG 10 or temporary GES 10 to sense and detect events associated with gastric disorders. In one embodiment, an accelerometer can detect the motion of IPG 10 within the abdominal region of patient 5. A specific pattern of motion can be associated with retching that can accompany vomiting. If IPG 10 is configured to detect a retching episode, it can record it as an event and as an intensity of motion. Such events can be timestamped.

In some embodiments, and on a regular basis, detected gastric disorder events can be communicated to a clinician who can use this information to monitor the patient's disease. IPG 10 may be configured to transfer such information via wireless communication (such as Bluetooth) to a clinician's programmer 80 during an in-office visit. Alternatively, IPG 10 may be configured to transmit the information to a patient's device (such as a cell phone, smart watch or base station, and/or patient controller/programmer 90) on a regular basis. This device can then upload the information to a database that is periodically reviewed by a clinician.

A clinician may choose to change electrical stimulation programming parameters based on the received information. For instance, if the number of episodes is too high over a given amount of time, the amount of electrical stimulation energy delivered to the patient may be increased by increasing amplitude, pulse width, stimulation duty cycle, frequency, etc. Alternatively, if symptoms are shown to be well controlled, the parameters may be adjusted to optimize battery life.

IPG 10 or temporary GES 10 may also be configured to operate in accordance with an algorithm which automatically adjusts gastric stimulation parameters based on detected gastric disorder events. For example, a clinician may set a threshold for events (e.g., 5 episodes a week). If the number of events exceeds the threshold, IPG 10 or temporary GES 10 can be configured to change to a new stimulation program parameter set that has been programmed by the clinician. By way of non-limiting illustrative example, IPG 10 or temporary GES 10 may be preprogrammed with 5 sets of parameters which can be selected from by the clinician. See, for example, FIG. 13.

Continuing to refer to FIG. 13, there is shown one embodiment of method/algorithm 700 for detecting and recording gastric disorder events in IPG or temporary GES 10. At step 701, multiple different sets of operating parameters for IPG or temporary GES 10 are determined or defined and loaded into a memory of IPG or temporary GES 10. These different sets of operating parameters can be configured to permit sensors on IPG or temporary GES 10 to detect or sense, and to record, different types of events, such as patient skin temperature, patient activity or non-activity, retching, vomiting, resting or active heart rate, sleep, body position (e.g., supine or standing), and so on. The detected events, depending on the context and the selected operating parameter set, may or may not be associated with gastric disorders (e.g., retching, normal sleep pattern, etc.). At step 703, one or more event thresholds are defined for given types of events, such as how long patient activity or non-activity lasts, how long the patient is sleeping or in a particular body position, the degree, amplitude or other characteristics of body movement associated with retching or vomiting, etc. At step 705, when an even has been detected, IPG or temporary GES 10 is configured to record data associated with the detected event. If the event threshold is exceeded as determined at step 707, IPG or temporary GES 10 changes to a new predetermined set of operating parameters. If not, IPG or temporary GES 10 continues to detect and record data associated with subsequent events.

According to one example of pseudocode not intended to be limiting, Table 3 below shows one embodiment of method/algorithm 700 and pseudocode corresponding thereto configured to sense and detect events associated with gastric disorders. According to the pseudocode of Table 3, functions for loading parameter sets, defining event thresholds, detecting events, recording event data, and adjusting stimulation parameters based on detected events are provided. The pseudocode of Table 3 also includes placeholders for functions that interact with device sensors and communicate with clinicians. These placeholders are intended to contain actual implementation details specific to the IPG 10, temporary GES 10, and/or computing device that is/are to be employed.

TABLE 3
One Embodiment of Pseudocode for Method/Algorithm 700

```
// Define the main function
function controlGESDeviceForGastricDisorders( ) {
  // Step 701: Load multiple sets of operating parameters
  loadOperatingParameters( )
  // Step 703: Define event thresholds
  defineEventThresholds( )
  // Monitor the patient continuously
  while (true) {
    // Step 705: Detect events and record data
    event = detectEvent( )
    If (event != null) {
      recordEventData(event)
      // Step 707: Check if event threshold is exceeded
      if (isEventThresholdExceeded(event)) {
        changeToNewOperatingParameters( )
      }
    }
    // Wait for a short period before checking again
    sleep(1 minute)
  }
}
// Load multiple sets of operating parameters into device memory
function loadOperatingParameters( ) {
  // Define and load parameter sets for various conditions
  parameterSets = [ { condition: ″retching″, pulseFrequency: high, cycleOnTime: high },
{ condition: ″vomiting″, pulseFrequency: medium, cycleOnTime: medium }, { condition:
″sleep″, pulseFrequency: low, cycleOnTime: low }, // Additional parameter sets can be
defined here ]
  loadIntoMemory(parameterSets)
}
// Define thresholds for various events
function defineEventThresholds( ) {
  eventThresholds = {
    ″retching″: { duration: 15 minutes, intensity: high },
    ″vomiting″: { duration: 10 minutes, intensity: medium },
    ″sleep″: { duration: 30 minutes, movement: low },
    // Additional thresholds can be defined here
  }
  saveEventThresholds(eventThresholds)
}
// Detect events using device sensors
function detectEvent( ) {
  // Check accelerometer and other sensors for events
  motionPattern = checkAccelerometer( )
  heartRate = checkHeartRate( )
  temperature = checkTemperature( )
  if (isRetchingPattern(motionPattern)) {
    return ( type: ″retching″, data: motionPattern }
  } else if (isVomitingPattern(motionPattern) {
    return { type: ″vomiting″, data: motionPattern }
  } else if (isSleepPattern(heartRate, temperature) {
    return { type: ″sleep″, data: { heartRate: heartRate, temperature: temperature } }
  }
  // Return null if no event detected
  return null
}
// Record data associated with the detected event
function recordEventData(event) {
  timestamp = getCurrentTime( )
  saveEventToMemory(event, timestamp)
  communicateEventToClinician(event, timestamp)
}
// Check if the detected event exceeds the defined threshold
function isEventThresholdExceeded(event) {
  threshold = getEventThreshold(event.type)
  return event.data.intensity >= threshold.intensity && event.data.duration >=
threshold.duration
}
// Change to a new set of operating parameters based on the event
function changeToNewOperatingParameters( ) {
  newParameters = selectNewParameters( )
  applyTherapyParameters(newParameters)
}
// Check if the detected motion pattern matches retching
function isRetchingPattern(motionPattern) {
  // Logic to determine if motion pattern matches retching
  return motionPattern.intensity > high && motionPattern.duration > 15 minutes
}
// Check if the detected motion pattern matches vomiting
function isVomitingPattern(motionPattern) {
  // Logic to determine if motion pattern matches vomiting
  return motionPattern.intensity > medium && motionPattern.duration > 10 minutes
}
// Check if the detected heart rate and temperature indicate sleep
function isSleepPattern(heartRate, temperature) {
  // Logic to determine if heart rate and temperature indicate sleep
  return heartRate < low && temperature < normal && checkBodyPosition( ) == ″supine″
}
// Communicate detected event to clinician
function communicateEventToClinician(event, timestamp) {
  // Logic to communicate event data to clinician via Bluetooth or other wireless method
  sendEventToClinician(event, timestamp)
}
// Placeholder function to load parameter sets into device memory
function loadintoMemory(parameterSets) {
  // Code to load parameter sets into device memory
}
// Placeholder function to save event thresholds
function saveEventThresholds(eventThresholds) {
  // Code to save event thresholds
}
// Placeholder function to get the current time
function getCurrentTime( ) {
  // Code to get the current time
  return new Date( )
}
// Placeholder function to check accelerometer data
function checkAccelerometer( ) {
  // Code to check accelerometer data
  return { intensity: getintensity( ), duration: getDuration( ) }
}
// Placeholder function to check heart rate
function checkHeartRate( ) {
  // Code to check heart rate
  return getHeartRate( )
}
// Placeholder function to check temperature
function checkTemperature( ) {
  // Code to check temperature
  return getTemperature( )
}
// Placeholder function to get the event threshold
function getEventThreshold(eventType) {
  // Code to get the threshold for the specified event type
  return eventThresholds[eventType]
}
// Placeholder function to save event data to memory
function saveEventToMemory(event, timestamp) {
  // Code to save event data to memory
}
// Placeholder function to send event data to clinician
function sendEventToClinician(event, timestamp) {
  // Code to send event data to clinician
}
// Placeholder function to select new parameters based on the event
function selectNewParameters( ) {
  // Logic to select new parameters
  return newParameters
}
// Placeholder function to apply therapy parameters to the device
function applyTherapyParameters(parameters) {
  // Code to apply therapy parameters to the GES device
}
// Placeholder function to get intensity of motion
function getintensity( ) {
  // Code to get Intensity of motion
}
// Placeholder function to get duration of motion
function getDuration( ) {
  // Code to get duration of motion
}
// Placeholder function to get heart rate
function getHeartRate( ) {
  // Code to get heart rate
}
// Placeholder function to get temperature
function getTemperature( ) {
  // Code to get temperature
}
// Placeholder function to check body position
function checkBodyPosition( ) {
  // Code to check body position
}
// Placeholder function to pause execution for a given duration
function sleep(duration) {
  // Code to pause execution
}
```

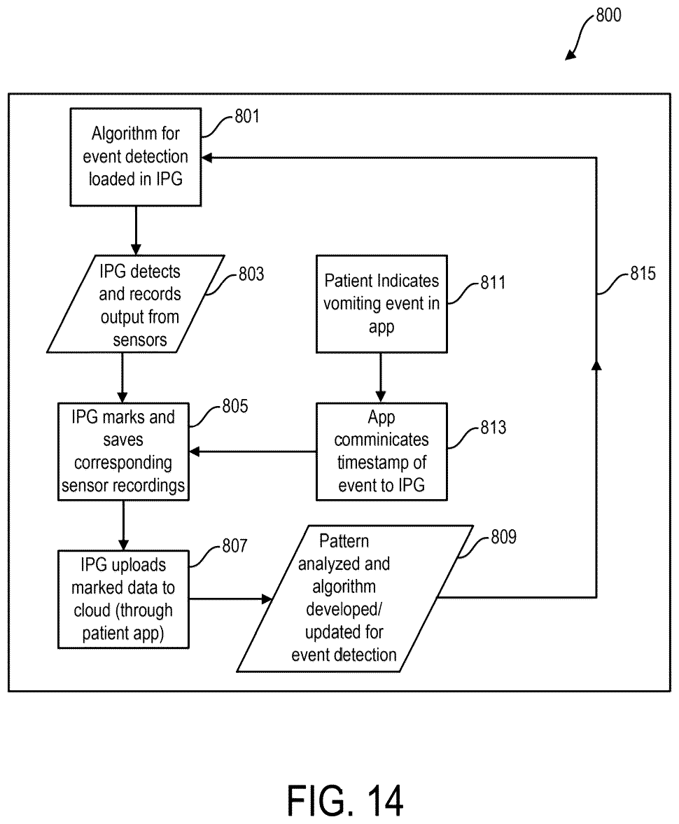

Referring now to method/algorithm 800 of FIG. 14, IPG 10 or temporary GES 10 can also be configured to optimize gastric disorder event detection for a particular individual. For instance, IPG 10 or temporary GES 10 may be configured to monitor its accelerometer (if included in IPG 10 or temporary GES 10) and record accelerometer data. In one embodiment, the recording may be on a memory loop for a specified time, for instance 10 minutes, to save memory. If the patient experiences a vomiting or retching episode, they can record the event on a handheld device (such as a phone app) that is in communication with IPG 10 or temporary GES 10, which then marks that pattern within its memory as a gastric disorder episode, and can save the episode or upload it to a patient device, and ultimately to a central location for physician review. This information can be used to optimize gastric disorder event detection thresholds within the algorithm, with IPG 10 then updated with an adjusted algorithm.

Continuing to refer to FIG. 14, there is shown one embodiment of method/algorithm 800 for detecting, recording and employing pattern recognition or other similar analytical techniques to improve gastric disorder event detection in IPG or temporary GES 10, where such analytical techniques can include determining trends, making predictions, and making decisions regarding previously-detected and/or newly-detected gastric disorder events, or combinations thereof. Many different types of pattern recognition algorithms, including decision trees, support vector machines, and neural networks, are contemplated for use in method/algorithm 800. Pattern recognition algorithms are recognized as a set of techniques used to identify patterns in data.

Nausea in patient 5 can be accompanied by heart rate changes and skin temperature changes. A temperature sensor in IPG 10 or operably connected thereto can be used to track local temperature changes, for example in the surface of IPG 10 or temporary GES 10. In one embodiment, patient heart rate can be detected using implanted electronics and ECG recording leads/electrodes. IPG 10 or temporary GES 10 can be trained to detect episodes of nausea, similar to the training method described above for vomiting episodes. Artificial intelligence algorithms can also be used to train IPG 10 to detect such nausea events with a high degree of accuracy. The detection of nausea events can also be used to optimize stimulation parameters as described above.

In still further embodiments, a patient's wearable device (such as a smart watch), can be used to detect a patient's temperature and heart rate and be used to track symptoms. Microphones and/or accelerometers can also be used to monitor for the sounds of vomiting and retching. These symptoms can be tracked and used to optimize parameters, either on their own, or in combination (e.g., communicate directly to the IPG) with on-board IPG sensors. An accelerometer may be employed to identify a motion pattern associated with vomiting. IPG programmed settings may also be modified using closed-loop feedback control methods and sensors, and a training algorithm can further be used to teach an IPG to detect vomiting.

Continuing to refer to FIG. 14, in method/algorithm 800, at step 801 method/algorithm 800 is loaded into a memory of IPG or temporary GES 10. At step 803. IPG or temporary GES 10 detects and records outputs from one or more sensors operably connected to IPG or temporary GES 10, including but not limited to temperature, accelerometer, motion, body position, acoustic, sleep, activity, and/or heart rate sensors. Step 803 can include detecting a gastric disorder event such as retching, vomiting, and so on, with or without receiving confirmatory input from patient 5 regarding the occurrence of a gastric disorder event. At step 805, IPG or temporary GES marks (e.g., time marks, geographical location mark, type of gastric disorder mark, relationship to other gastric disorder events that have been marked, etc.) the sensor recordings from step 803. At step 811, patient 5 can indicate that a vomiting or other gastric disorder event has occurred by, for example, pressing a button or switch operably connected to IPG or temporary GES 10, or by providing an input to an app loaded in controller/programmer 90 and operably connected to IPG or temporary GES 10. At step 813, the timestamp or other data associated with the gastric disorder event input by patient 5 at step 811 is communicated to IPG or temporary GES 10. At step 807, IPG or temporary GES 10 uploads or transfers the time- or other-marked data from step 805 to the cloud, an internal memory in IPG or temporary GES 10, a local or remote computer, database or server, or to controller/programmer 80 and/or 90, or to a computing device for further processing and analysis at step 809. At step 809, the data from steps 805 and 807 are analyzed and processed to detect patterns and characteristics in the gastric disorder data that can then be used in steps 801 through 809 to improve subsequent gastric disorder event detection and analysis occurring in method/algorithm 800. Artificial intelligence techniques can be employed in method/algorithm 800 to improve gastric disorder event detection and analysis.

According to one example of pseudocode not intended to be limiting, Table 4 below shows one embodiment of method/algorithm 800 and pseudocode corresponding thereto configured to record and employ pattern recognition or other similar analytical techniques to improve the detection of gastric disorder events. According to the pseudocode of Table 4 method/algorithm 800 is configured to detect, record, and optimize gastric disorder event detection using various sensors and pattern recognition techniques. The pseudocode shown in Table 4 includes functions for loading method/algorithm 800 into memory, detecting, and recording sensor outputs, marking data, recording patient-indicated events, uploading data, and analyzing data to improve gastric disorder event detection. The pseudocode shown in Table 4 also includes placeholders for functions that interact with sensors and apply pattern recognition algorithms, which are implemented based on the specific details of the particular IPG 10, temporary GES device 10, sensors, and/or computing device that is/are to be employed.

TABLE 4
One Embodiment of Pseudocode for Method/Algorithm 800

```
// Define the main function
function optimizeGastricDisorderEventDetection( ) {
  // Step 801: Load the algorithm into IPG memory
  loadAlgorithmToMemory( )
  while (true) {
    // Step 803: Detect and record sensor outputs
    sensorData = detectAndRecordSensorOutputs( )
    // Step 805: Mark the recorded data
    markedData = markSensorData(sensorData)
    // Step 811: Check for patient input indicating an event
    If (patientindicatesEvent( ) {
      // Step 813: Record the event indicated by the patient
      recordPatientIndicatedEvent( )
    }
    // Step 807: Upload marked data for further processing
    uploadMarkedData(markedData)
    // Step 809: Analyze the data to detect patterns and improve detection
    analyzeDataToImproveDetection(markedData)
    // Wait for a short period before checking again
    sleep(1 minute)
  }
}
// Load the algorithm into IPG memory
function loadAlgorithmToMemory( ) {
  // Code to load the algorithm into the memory of IPG or temporary GES 10
}
// Detect and record sensor outputs
function detectAndRecordSensorOutputs( ) {
  // Collect data from various sensors
  temperature = getTemperature( )
  accelerometer = getAccelerometerData( )
  motion = getMotionData( )
  bodyPosition = getBodyPosition( )
  acoustic = getAcousticData( )
  sleep = getSleepData( )
  activity = getActivityData( )
  heartRate = getHeartRate( )
  return {
    temperature: temperature,
    accelerometer: accelerometer,
    motion: motion,
    bodyPosition: bodyPosition,
    acoustic: acoustic,
    sleep: sleep,
    activity: activity,
    heartRate: heartRate
  }
}
// Mark the recorded sensor data
function markSensorData(sensorData) {
  timestamp = getCurrentTime( )
  geographicalLocation = getGeographicalLocation( )
  eventType = determineEventType(sensorData)
  return {
    data: sensorData,
    timestamp: timestamp,
    location: geographicalLocation,
    eventType: eventType
  }
}
// Check if the patient indicates an event
function patientindicatesEvent( ) {
  // Code to check if the patient has indicated a gastric disorder event via a handheld device
  return checkPatientinput( )
}
// Record the event indicated by the patient
function recordPatientindicatedEvent( ) {
  // Code to record the patient-indicated event with its timestamp and other details
  eventTimestamp = getCurrentTime( )
  savePatientEvent(eventTimestamp)
}
// Upload marked data for further processing
function uploadMarkedData(markedData) {
  // Code to upload the marked data to the cloud or other storage for analysis
  uploadDataToServer(markedData)
}
// Analyze the data to detect patterns and improve detection
function analyzeDataToimproveDetection(markedData) {
  // Use AI techniques to analyze data and detect patterns
  patterns = applyPatternRecognitionAlgorithms(markedData)
  // Use detected patterns to improve the algorithm
  updateAlgorithmWithNewPatterns(patterns)
}
// Placeholder function to get the current time
function getCurrentTime( ) {
  // Code to get the current time
  return new Date( )
}
// Placeholder function to get geographical location
function getGeographicalLocation( ) {
  // Code to get the geographical location
  return getLocation( )
}
// Placeholder function to determine event type from sensor data
function determineEventType(sensorData) {
  // Logic to determine the type of gastric disorder event from sensor data
  return identifyEventType(sensorData)
}
// Placeholder function to check patient input
function checkPatientInput( ) {
  // Code to check for patient input indicating an event
  return checkHandheldDeviceInput( )
}
// Placeholder function to save patient event
function savePatientEvent(eventTimestamp) {
  // Code to save the patient-indicated event with timestamp
}
// Placeholder function to upload data to server
function uploadDataToServer(data) {
  // Code to upload data to a server or cloud storage
}
// Placeholder function to apply pattern recognition algorithms
function applyPatternRecognitionAlgorithms(data) {
  // Code to apply AI techniques to analyze data and detect patterns
  return recognizePatterns(data)
}
// Placeholder function to update the algorithm with new patterns
function updateAlgorithmWithNewPatterns(patterns) {
  // Code to update the algorithm with newly detected patterns
}
// Placeholder functions to get sensor data
function getTemperature( ) {
  // Code to get temperature data
}
function getAccelerometerData( ) {
  // Code to get accelerometer data
}
function getMotionData( ) {
  // Code to get motion data
}
function getBodyPosition( ) {
  // Code to get body position data
}
function getAcousticData( ) {
  // Code to get acoustic data
}
function getSleepData( ) {
  // Code to get sleep data
}
function getActivityData( ) {
  // Code to get activity data
}
function getHeartRate( ) {
  // Code to get heart rate data
      }
```

Existing systems typically comprise open-loop (i.e., non-feedback control-based) systems, and optimization of programming parameters requires patients to track events and provide subjective feedback to a clinician. Programming is often done in follow-up sessions. The improvements described and disclosed above and/or herein provide much quicker and more efficient ways to deliver optimized therapy to a patient using feedback control methods and associated sensors.

See also the following publications, each of which is hereby incorporated by reference into the specification of the present patent application, each in its entirety: (1) Chen, J. D., Qian, L., Ouyang, H., Yin, J., Disorders, E. N., & Group, P. (2003), “Gastric electrical stimulation with short pulses reduces vomiting but not dysrhythmias in dogs,” Gastroenterology, 124 (2), 401-409; (2) Abrahamsson, H. (1973), “Vagal relaxation of the stomach induced from the gastric antrum,” Acta Physiologica Scandinavica, 89 (3), 406-414; AND (3) Song, G. Q., Zhu, H., Lei, Y., Yuan, C., Starkebaum, W., Yin, J., & Chen, J. D. (2015), “Gastric electrical stimulation optimized to inhibit gastric motility reduces food intake in dogs,” Obesity Surgery, 25 (6), 1047-1055.

Note that in some embodiments the various methods and algorithms described above may be carried out not only by IPGs 10 or temporary GESs 10, but also by other types of implantable or external pulse generators, including miniaturized IPGs/GESs such as capsule 900, various embodiments of which are described in further detail below.

The methods, systems, devices and components described above may further include one or more of the following. According to some embodiments, there is provided a method of electrically stimulating a portion of a patient's stomach to treat one or more gastrointestinal disorders of the patient, the method comprising implanting an implantable pulse generator (IPG) in or near the stomach of the patient; over a first predetermined period of time, generating first electrical stimulation signals in the IPG, the first electrical stimulation signals having one or more frequencies ranging between about 2 Hz and about 120 Hz, one or more pulse widths ranging between about 100 μsec. and about 10 msec., one or more amplitudes ranging between about 0.1 mA and about 20 mA; providing, over at least a portion of the first predetermined period of time, the first electrical stimulation signals through at least one medical electrical lead and one or more electrodes thereof to a portion of the stomach of the patient; over a second predetermined period of time, generating second electrical stimulation signals in the IPG, the second electrical stimulation signals having one or more of frequencies, pulse widths and amplitudes less than one or more of the corresponding frequencies, pulse widths and amplitudes of the first electrical stimulation signals; providing, over at least a portion of the second predetermined period of time, the second electrical stimulation signals through the at least one medical electrical lead and the one or more electrodes thereof to the portion of the stomach of the patient; as one or more of the frequencies, pulse widths, and amplitudes of the electrical stimulation signals provided to the patient continue to be successively reduced, determining one or more of the frequency, pulse width, and amplitude parameters of the electrical stimulation signal wherein efficacy in treating the one or more gastrointestinal disorders of the patient is reduced or lost, and on the basis of the frequency, pulse width, and amplitude parameters determined to be associated with reduced or lost efficacy in treating the one or more gastrointestinal disorders of the patient, generating and providing to the patient chronic electrical stimulation signals having one or more of increased frequency, pulse width, and amplitude parameters compared to the frequency, pulse width, and amplitude parameters determined to be associated with substantially reduced or lost efficacy in treating the one or more gastrointestinal disorders of the patient; wherein the one or more gastrointestinal disorders of the patient include at least one of nausea, vomiting, early satiety, postprandial fullness, and abdominal pain and the method results in more rapid and accurate programming and determination of gastric stimulation parameters for the patient compared to conventional gastric stimulation programming techniques or methods.

Such a method may further comprise one or more of: (i) after the second electrical stimulation signals have been provided, over a third predetermined period of time, generating third electrical stimulation signals in the IPG, the third electrical stimulation signals having one or more of frequencies, pulse widths and amplitudes less than one or more of the corresponding frequencies, pulse widths and amplitudes of the second electrical stimulation signals, and providing, over at least a portion of the third predetermined period of time, the third electrical stimulation signals through the at least one medical electrical lead and the one or more electrodes thereof to the portion of the stomach of the patient; (ii) wherein the first electrical stimulation signals have at least one of frequencies ranging between about 10 Hz and about 110 Hz and pulse widths ranging between about 200 μsec. and about 500 μsec.; (iii) wherein at least one of the first electrical stimulation signals and the second electrical stimulation signals are provided with a duty cycle ranging between about 01. seconds on and about 10 seconds on, and between about 1 second off and about 20 seconds off; (iv) wherein at least one of the first electrical stimulation signals and the second electrical stimulation signals are provided according to a duty cycle ranging between: (a) about 0.1 seconds on and about 5 seconds off; (b) about 1 second on and about 4 seconds off; (c) about 2 seconds on and about 3 seconds off, and (d) about 4 seconds on and about 1 second off; (v) wherein the chronic electrical stimulation signals are provided to the portion of the patient's stomach at a reduced frequency when the patient is one more of detected, and is scheduled, to be sleeping or resting, thereby to reduce the power consumption of the IPG; (vi) wherein the chronic electrical stimulation signals are not provided to the portion of the patient's stomach when the patient is detected to be sleeping or resting, thereby to reduce the power consumption of the IPG; (vii) wherein the chronic electrical stimulation signals are not provided to the patient when the patient is detected to be sleeping or resting or when the patient is scheduled to undergo a nocturnal cycling protocol, thereby to reduce the power consumption of the IPG; (viii) wherein the IPG includes or comprises, or is operably connected to, one or more of a sleep detector, an accelerometer, and a patient position detector; (ix) wherein the patient not does have a gastric motility disorder; (x) wherein the patient has a gastric motility disorder; (xi) wherein the gastric motility disorder is one of delayed gastric emptying and impaired fundic accommodation; (xii) wherein at least some of the chronic electrical stimulation signals provided to the patient are configured to mimic at least one of gastric slow wave activity and gastric spike activity indicative of smooth muscle depolarization or contraction, thereby to treat delayed gastric emptying in the patient; (xiii) wherein at least some of the chronic electrical stimulation signals provided to the patient are configured to mimic a desired fundic accommodation response in the patient: (xiv) wherein at least some of the chronic electrical stimulation signals provided to the patient to mimic a desired fundic accommodation response are provided continuously or in bursts; (xv) wherein at least some of the chronic electrical stimulation signals provided to the patient to mimic a desired fundic accommodation response are provided at the onset of a meal taken by the patient; (xvi) wherein at least some of the chronic electrical stimulation signals provided to the patient to mimic a desired fundic accommodation response are terminated when a meal taken by the patient ends: (xvi) wherein at least some of the chronic electrical stimulation signals provided to the patient to mimic a desired fundic accommodation response are controlled, initiated, modified, or terminated by a patient controller, a physician controller, a sensor, or a timer; (xvii) wherein the patient can use an app and corresponding external device to record one or more feelings of satiety, fullness, vomiting and nausea; (xviii) wherein the electrical stimulation signals are provided by a gastric stimulator comprising a stimulation module and at least one stimulation electrode operably connected thereto and associated therewith; (xix) wherein the gastric stimulator further comprises electrical stimulation electronics and a power source associated therewith; (xx) wherein the gastric stimulator further comprises communication electronics configured to permit wireless communication and control or programming thereof from outside the body of the patient after the stimulator has been in the patient; (xxi) wherein the implantable stimulation module and the at least one electrode are contained in, or each form a portion of, a capsule or housing; (xxii) wherein the gastric stimulator further comprises one or more of a rechargeable battery, a primary battery, and a power source; (xxiii) wherein the at least one stimulation electrode forms a portion of a medical electrical lead, the medical electrical lead being operably connected to the implantable stimulation module and configured to deliver electrical stimulation signals from the module to the lead; (xxiv) wherein the lead comprises at least one return or ground electrode; (xxv) wherein the lead comprises multiple stimulation electrodes; (xxvi) wherein the electrodes are at least one of unipolar, bipolar and multi-polar; (xxvii) wherein a biodegradable or releasable link is disposed between at least a portion of the lead and the stimulation module, and at least one of the lead and the stimulation module is configured to be released from attachment to or positioning within the tunnel, the first space, the second space, or the submucosal layer, and then to pass harmlessly through the patient's digestive system after the biodegradable link has dissolved or the link has been released after a predetermined period of time has passed or upon receipt of a command by the stimulator from an external communication device; (xxviii) wherein the stimulation module comprises at least one return or ground electrode; (xxix) wherein the gastric stimulator is a temporary gastric stimulator implanted endoscopically in a submucosal layer or space in the patient's stomach; (xxx) wherein the temporary gastric stimulator further or one or more portions thereof such as a lead portion comprises at least one fixation member or feature configured to affix the stimulator or portion thereof to the submucosal layer; (xxxi) wherein the fixation member or feature comprises one or multiple ones of a tine, a helical fixation wire, a staple, and a fixation pin; (xxxii) wherein the method further comprises one or more G POEM steps, techniques or methods, and (xxxiii) further comprising the temporary gastric stimulator or one or more portions thereof, being configured to be passed safely through the patient's digestive tract after being released from the stomach by the temporary gastric stimulator or one or more portions thereof being released or through the action of a biodegradable link or connection dissolving.

### 2. Temporary Gastric Electrical Stimulation

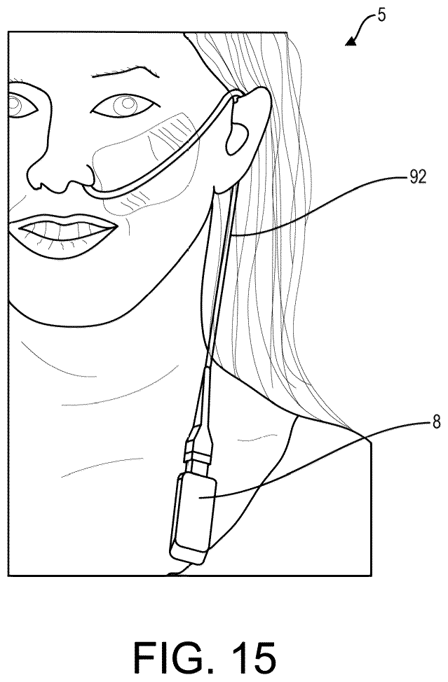

The most common method of performing temporary gastric stimulation is by using a temporary pacing lead clipped to the mucosal lining of the stomach. Lead placement and fixation in such a procedure is typically performed via an endoscope. The lead body is routed through the patient's nose or mouth and attached to a stimulator worn on the outside of the body. (See, for example, Yinala S, Batista O, Goyal A, et al., “Temporary gastric electrical stimulation with orally or PEG-placed electrodes in patients with drug refractory gastroparesis,” Gastrointest Endosc 2005; 61:455-61.) FIG. 15 illustrates a similarly placed pH meter readout device 8. Percutaneous methods have also been described. (See, for example, Elfvin A, Andersson S, Abrahamsson H, Edebo A, Simrén M, Lönroth H., “Percutaneous implantation of gastric electrodes-a novel technique applied in animals and in patients,” Neurogastroenterol Motil. 2007 February; 19 (2): 103-9. doi: 10.1111/j.1365-2982.2006.00858.x. PMID: 17244164.)

Some gastro-intestinal (GI) doctors and patients would like to be able to trial gastric electrical neurostimulation prior to the implant of a permanent system. A permanent system is usually implanted by placing lead electrodes into the stomach wall via a laparoscopic procedure. Many patients are unwilling to have this procedure done without knowing if it will provide relief.

In addition, some gastroparesis patients do not have an immediate sensation related to the program settings of a temporary or permanent gastric electrical stimulator. It may take some time to determine if the patient's symptoms are improving as a result of the delivery of gastric electrical stimulation regime. In many cases, it would be desirable to provide gastric electrical stimulation to a patient for a few days via a minimally invasive approach to determine if the stimulation can provide symptom relief prior to incurring the expense and undergoing the invasiveness of a chronic implant.

In some embodiments, there are provided systems, devices, components and methods for temporary gastric electrical stimulation and associated methods of implantation within a patient's stomach. Some embodiments provide a more patient friendly alternative to having a wired device disposed through a patient's nose or mouth, and can provide stable temporary implants.

In some embodiments, a patient can experience temporary gastric electrical stimulation prior to a permanent implant, without a wire running through the patient's nose or mouth for the duration of the trial. By placing all, or part, of the system within, for example, a submucosal tunnel that has been created endoscopically, stable fixation is created. In some embodiments, a temporary gastric electrical stimulator, or portions thereof, can be retrieved using an endoscope at the end of the trial. In some embodiments where a temporary gastric electrical stimulator system employs a rechargeable or wirelessly powered device, the system can be left in the patient indefinitely, which cannot be done using a conventional wired system in use currently. If bioabsorbable or degradable fixation means are used, the temporary gastric electrical stimulator device, or portions thereof, can be configured to just pass through the digestive tract of the patient after the temporary stimulation period has ended. In such a case, the patient is not required to come back to the medical office to have the lead or device removed.

In some embodiments, a neurostimulator is configured to be at least partially inserted into an endoscopically created submucosal space in the digestive tract. Such a method of implant can comprise creating a submucosal “3rd space” and inserting all or part of a neurostimulator into the space, some embodiments of which are described in detail below. Biodegradable portions of a neurostimulator or temporary gastric electrical stimulator can also be provided and configured to release at least part of the stimulator from the patient's stomach, allowing it to pass out of the patient through the digestive tract.

In one embodiment, a surgical procedure is provided in which a temporary gastric electrical stimulator, or portions thereof, are implanted via an endoscope and “pinned” to the mucosal lining of the stomach, such as by using a mechanism similar to that employed in the Medtronic Bravo™ pH sensor, more about which is said below.

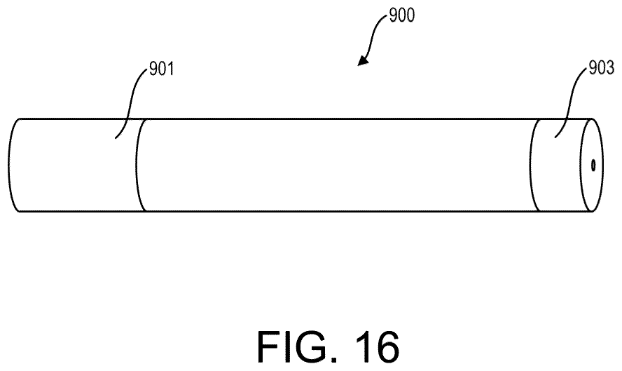

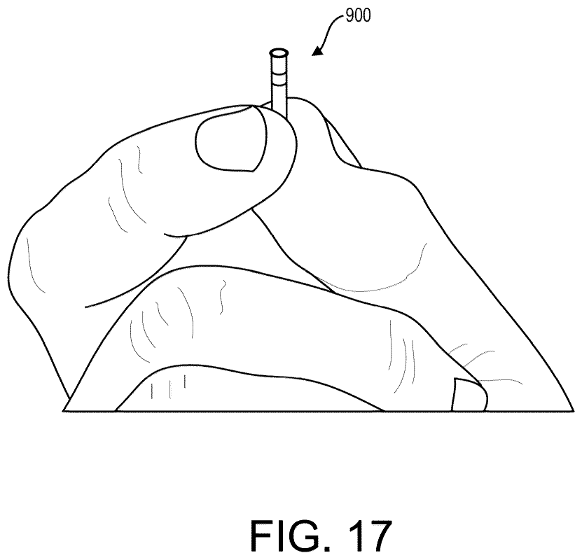

In one embodiment, a temporary gastric electrical stimulator is configured as a small capsule 900, as shown in FIG. 16. Such a temporary gastric electrical stimulator can comprise miniature electronics disposed inside the capsule and two electrodes 901 and 903 disposed on the outside of capsule 900, as shown in FIG. 16. FIG. 17 shows an illustrative scale of the stimulator capsule 900 shown in FIG. 16. Appropriately configured, stimulator capsule 900 can include all, most or portions of the circuitry described above in connection with FIGS. 3-6.

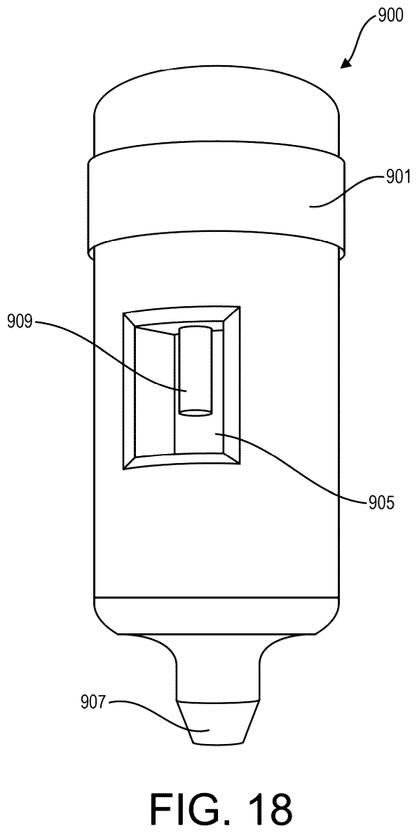

FIG. 18 shows another embodiment of a capsule or mini-IPG 900 comprising electrode ring or band 901, fixation pin 909 disposed in recess or tissue window 905, and vacuum attachment 907. In the embodiment illustrated in FIG. 18, temporary gastric electrical stimulator 900 contains electronics to generate a stimulation pulse, a battery to power the device and at least two electrodes. Capsule 900 can also contain components to permit wireless communication so that programming parameters can be defined and adjusted post-implantation. In other embodiments, wireless communication with the temporary gastric stimulator is absent. For example, if programming is required at the time of implantation with only a one or two week duration of temporary electrical stimulation required, direct cable programming to the stimulator can be employed pre-implant to set high stimulation levels, which can then be used to reveal whether GES reduces unwanted symptoms; appropriate stimulation thresholds can be determined later, thereby eliminating the costs associated with wireless programming. Eliminating features typically associated with much larger IPGs 10 from a temporary capsule stimulator 900 can also reduce device complexity and volume otherwise occupied by wireless circuitry, permitting a smaller device.

In FIG. 18, and according to one embodiment, one electrode 909 is a fixation pin, and the other electrode 901 is a band on the outside of the capsule. Capsule 900 can be configured to be mounted in a delivery catheter, where the catheter has a proximal port that can be attached to a vacuum on the connector side, and a distal port configured to connect to a vacuum attachment point in capsule 900. Capsule 900 can be configured for delivery through the mouth and esophagus to the stomach of patient 5, and positioned with tissue window 905 disposed against the stomach lining. While being delivered, fixation pin 909 is retracted within capsule 900 to secure it in position against the interior gastric wall. Once positioned, a vacuum is pulled, which causes tissue to enter the tissue window 905. The fixation pin 909 is then deployed into the tissue, which holds capsule 900 in place. In one embodiment, when the vacuum is released from the catheter, capsule 900 is released from the catheter. Other capsule fixation and release mechanisms are also contemplated.

Capsule 900 can be configured to remain in place for several days or weeks until the mucosa slough off due to tissue trauma induced by pin 909. Capsule 900 can further be configured to be safe to pass through the patient's digestive track when it does slough off from the mucosal wall. In the meantime, capsule 900 can stimulate stomach 200 so that patient 5 undergoes the desired gastric electrical stimulation treatment and therapy.

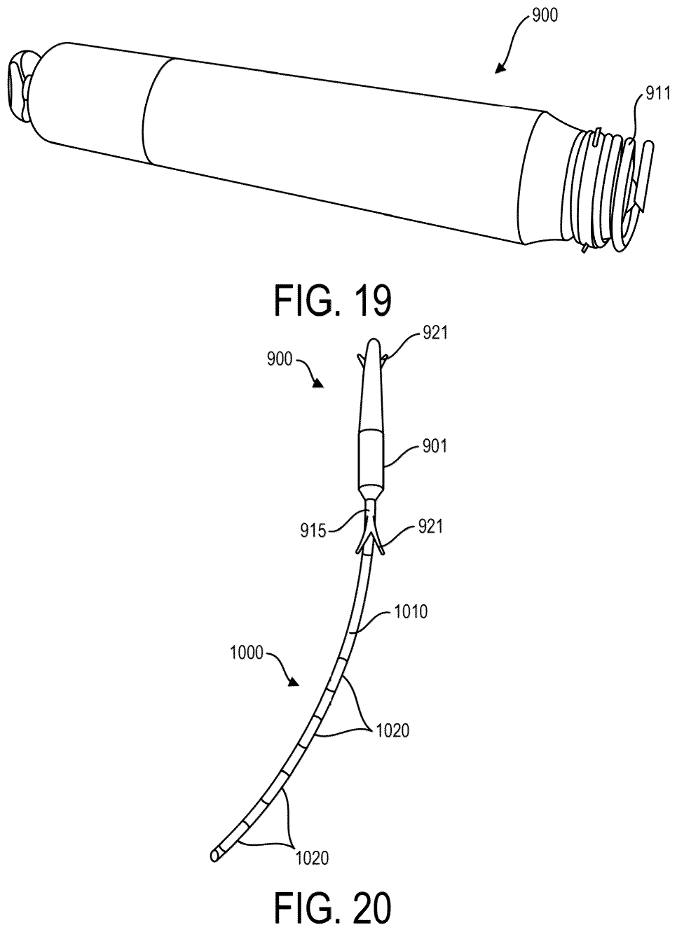

In some embodiments, a temporary gastric stimulator may also comprise a small primary cell battery, a rechargeable battery, or be powered wirelessly from outside the body using, for example, inductive power transfer means. Capsule 900 shown in FIGS. 16-18 may have two or more electrodes 901/903/909 disposed on the exterior of capsule 900. Pin 909 may or may not be electrically active in this case. While electrodes are shown in FIGS. 16-18 as bands or pins, electrodes can also have circular, elliptical, square, rectangular or other shapes. Alternatively, a helix or screw 911 may be used to affix stimulator 900 to the stomach tissue, similar to a leadless pacemaker, as shown in FIG. 19.

The device shown in FIG. 19 can be configured to be unscrewed and retrieved endoscopically. Alternatively, screw 911 can be protected by a shroud or cover, or be spring loaded, such that helix or screw 911 is covered when capsule 900 detaches, protecting the digestive tract from the tip of helix or screw 911.

The device shown in FIG. 19, and other embodiments of temporary gastric stimulators described herein, may also be configured to sense electrogastrogram (EGG) signals, and to use telemetry to communicate EGGs to an external device such as controller/programmers 80 and/or 90, or to a smartwatch, a smart phone via an app, or a tablet. Such a device may also be configured to use ECG signals to auto-calibrate electrical stimulation amplitudes to adjust and/or ensure appropriate stimulation levels.

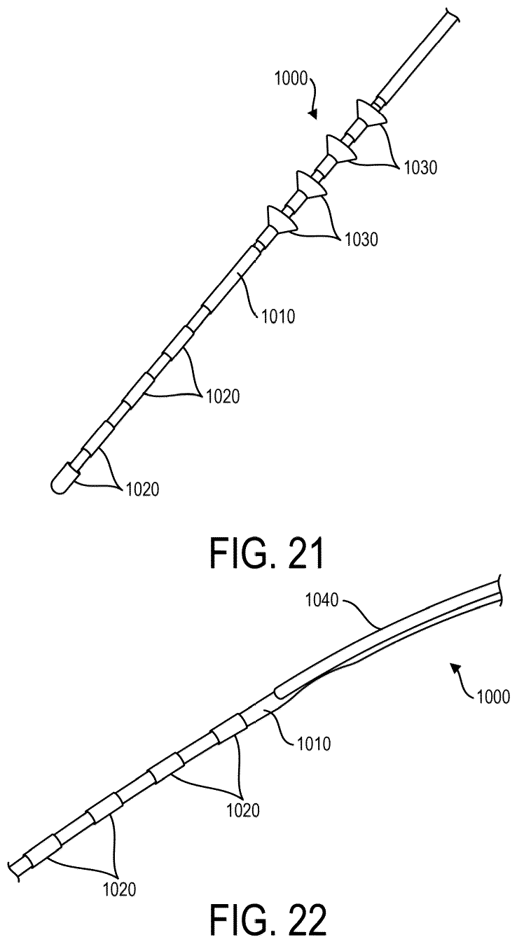

Alternatively, in another embodiment shown in FIG. 20, stimulator 900 may comprise an attached set of electrodes 1020 disposed along lead 1000 extending from IPG/capsule/stimulator 900, along with one or more electrodes 901 disposed on stimulator 900's case, body or capsule. Stimulator/capsule 900 fixation elements or features 921 may also be included in stimulator/capsule 900. In one embodiment, the assembly can comprise one or more electrodes 1020 on lead portion 1000, and one or more electrodes on the stimulator 900. Additionally, and in some embodiments, a biodegradable link 915 is disposed between stimulator 900 and lead 1000, which is configured to permit stimulator 900 to separate from lead 1000 and pass through patient 5's body after a predetermined period of time period has passed following implantation.

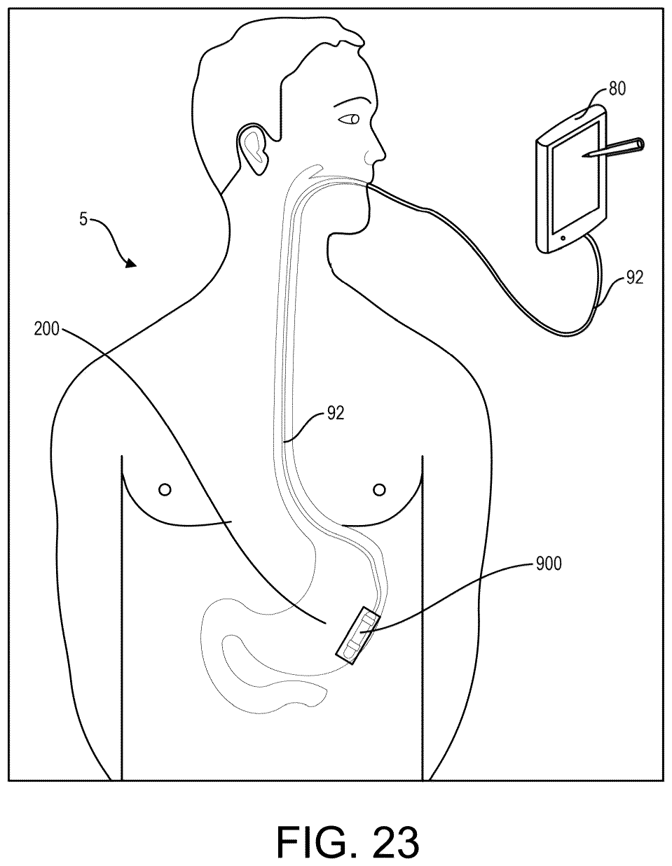

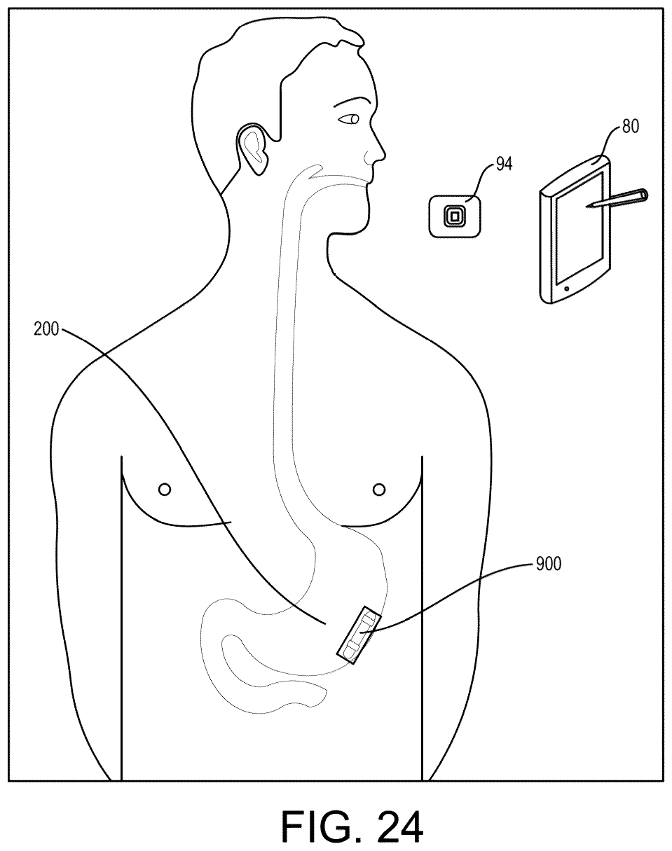

Lead 1000 of such a temporary gastric electrical stimulator 900 may also comprise fixation features configured to hold lead 1000 in place. See, for example, lead fixation tines or features 1030 in FIG. 21. Other types of lead fixation elements or features 1030 are also contemplated, such as tines, protrusions, bumps, collapsible collars, retractable/deployable rings or pins, helical screws, etc. In another embodiment shown in FIG. 22, a receiving coil 1040 and associated stimulation and communication electronics are built into lead body 1010.

In yet another embodiment, fixation elements or features 1030 are located on lead body 1010. In cases where fixation elements or features 1030 are employed in lead 1000 or lead body 1010, such fixation elements or features 1030 can be biodegradable, permitting the temporary gastric stimulator, or portion(s) thereof, to be released after a predetermined period of time after stimulator implant has passed or in response to a received communication signal provided by a health care provider.

There are now described still further embodiments of implantable temporary gastric electrical stimulators (temporary GESs), and methods, systems, devices and components associated therewith.

As described in some detail above, dysfunction of the gastric muscles or the mechanisms associated with controlling gastric motility can lead to gastroparesis. The disease gastroparesis is typically defined as a syndrome indicative of delayed gastric emptying, but is typically not associated with mechanical obstruction. Symptoms of gastroparesis usually include nausea, vomiting, bloating, abdominal pain, and early satiety. Initial treatment for these patients may include modification of diet and/or drugs.

If dietary changes or drugs are not successful in treating a patient's gastroparesis, a surgically implanted GES system can be a treatment alternative for conditions such as chronic intractable (drug refractory) nausea, vomiting secondary to gastroparesis of diabetic patients, and idiopathic etiology in patients typically aged 18 to 70 years. The electrodes for a permanent IPG 10 can be implanted in the greater curvature of the corpus region of the stomach (also considered the main gastric pacemaker region). Current technology requires that a permanent IPG system be implanted as a screening device to determine whether a patient is a potential candidate for GES therapy.

With presently available prior art technology for GES, a lead 100 and IPG 10 are implanted by a surgeon, typically using a laparoscopic procedure. Two leads 100 each with a single electrode are implanted on the circular muscle layer of the stomach about 10 mm apart. See, for example, FIG. 1. Patient screening and therapy assessment are then performed using such a system.

In one embodiment, and according to some embodiments described and disclosed herein, a temporary GES is implanted on the inner wall and mucosal layer of the stomach. This can be accomplished by a gastroenterologist using an endoscopic procedure, which is relatively simple and of low risk.

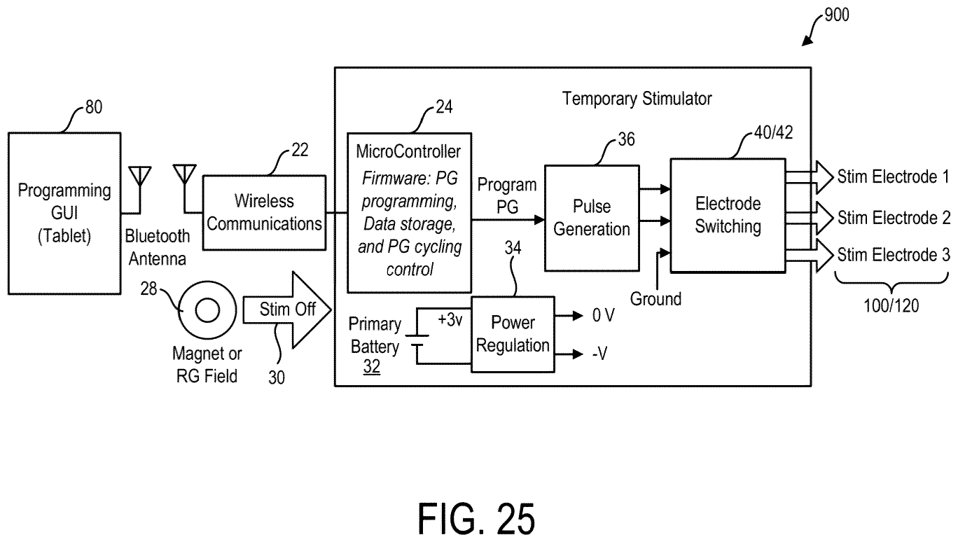

FIG. 23 shows one embodiment of such a procedure and system, and illustrates the implantation location of a temporary stimulator 900 on or beneath the mucosal wall of the stomach, and near where a permanent IPG 10 might otherwise be implanted (e.g., corpus antrum). In one embodiment, temporary GES 900 is configured to receive programming and other communication signals from an external controller or programmer (for example a tablet device) via cable 92. Clinician controller/programmer 80 can be configured to communicate with temporary GES 900 using a direct hard-wired connection provided by cable 92 or through wireless means.

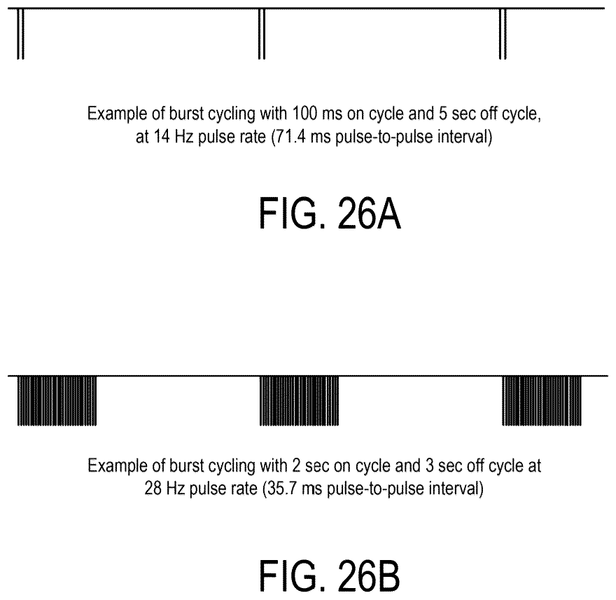

In one embodiment, once temporary GES 900 has been configured for initial operation, cable 92 providing a hard-wired connection (see FIG. 23) can be detached from the temporary GES (see FIG. 24 below), which has been secured to stomach 200. In some embodiments, the distally located port by which cable 92 is connected to temporary GES 900 is self-sealing when removed from temporary GES 900, thus establishing a barrier to the surrounding gastric and tissue environment. Once cable 92 has been removed from temporary GES 900, or while cable 92 is still operably connected to temporary GES 900, wireless communication (e.g., using Bluetooth or other wireless communication means) with temporary GES 900 may then be initialized or effected using Bluetooth module 94 and/or controller/programmer 80 or 90, where GES is equipped with appropriate wireless receiver circuitry. In another embodiment, Bluetooth module 94 can be connected to a proximal portion of programming cable 92 routed through the patient's esophagus, and configured to communicate an on-board Bluetooth module located in controller/programmer 90. As with the hardwired configuration, once temporary GES 900 has been appropriately configured, cable 92 and Bluetooth module 94 can be detached from cable 92 and temporary GES 900, and wireless communication between temporary GES 900 and controller/programmer 80 and/or 90 or with Bluetooth module 94 (see FIG. 24).

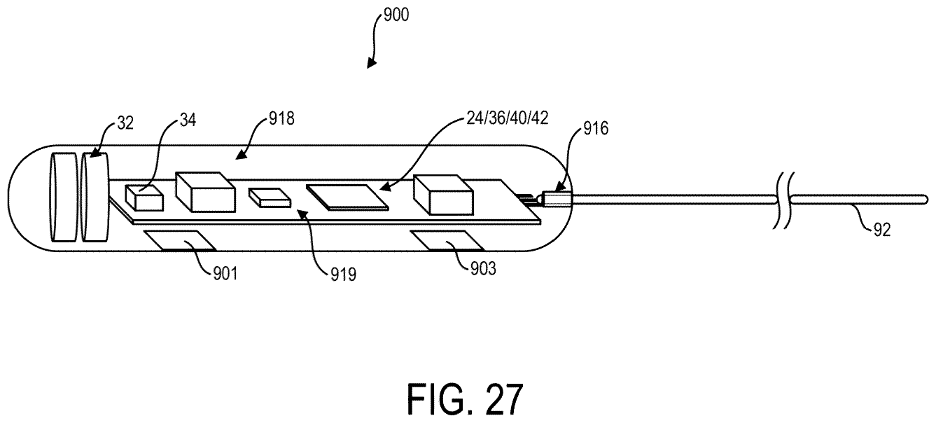

FIG. 25 illustrates one embodiment of a circuit block diagram for temporary GES 900. The temporary GES system can be configured to include a temporary GES 900, an external clinician's programmer or controller 90, and a magnet control device 28. Clinician controller/programmer 90 can be configured to communicate with temporary GES 900 through wireless communications (e.g., Bluetooth), thereby to provide instructions for parametric operational and other settings of temporary GES 900, and for retrieving measurement and other data from temporary GES 900.

Additionally, GES 900 can be configured to include wireless inductive battery charging circuitry, which permits the battery or batteries 32 of GES 900 to be recharged by inductive means from outside the body of patient 5, as is well known in the art. This of course requires that battery or batteries 32 of GES 900 be rechargeable or secondary batteries. The ability to recharge battery or batteries 32 of GES 900 by inductive and wireless means can be particularly important in cases where GES 900 is employed for electrical stimulation of stomach 200 within patient 5 for extended periods of time (e.g., more than several weeks or months), and where GES 900 would be considered to be a permanent or semi-permanent implant.

As described above, temporary GES 900 can include a separate wireless communications module, which can be configured to communicate with an external instrument such as clinician controller/programmer 90 and/or patient controller/programmer 80. Temporary GES 900 can also include a microcontroller or other CPU module 24 which is configured to receive information from and transmit information to the wireless communications module 22. The microcontroller module 24 may also be configured to communicate with the pulse generation module 36 to enable the setting of operational parameters such as electrode selection, stimulation frequency, pulse width, amplitude, pulse morphology, timing, and so on. The electrode switching module 40/42 can be configured to permit the selection of electrodes 1 for the delivery of stimulation pulses (e.g., cathodes) or for ground (e.g., anodes). Power for temporary GES 900 can be provided by a primary or secondary battery 32 via a power regulation module 34. The pulse generation module 36 can also be configured to provide stimulation pulses for operational performance measurements (e.g., lead impedance).

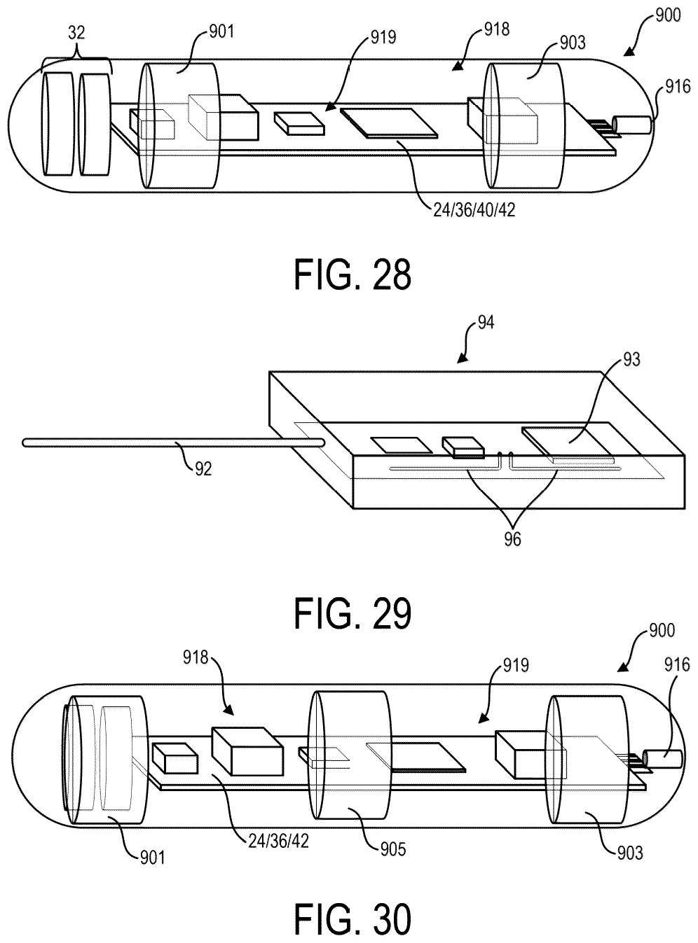

In one embodiment, a method for delivering GES neurostimulation pulses for the treatment of gastroparesis is to program output amplitude (e.g., stimulation current or voltage) stimulation pulse width, stimulation pulse rate (frequency), and burst pulse timing cycles. A burst of stimulation pulses with a specified repetitive interval (e.g., 5 seconds) and with a cycle On interval (e.g., 1 second), followed by a cycle Off interval (e.g., 4 seconds), which in combination establishes a burst duty cycle. See, by way of illustrative but non-limiting examples, FIGS. 26A and 26B.

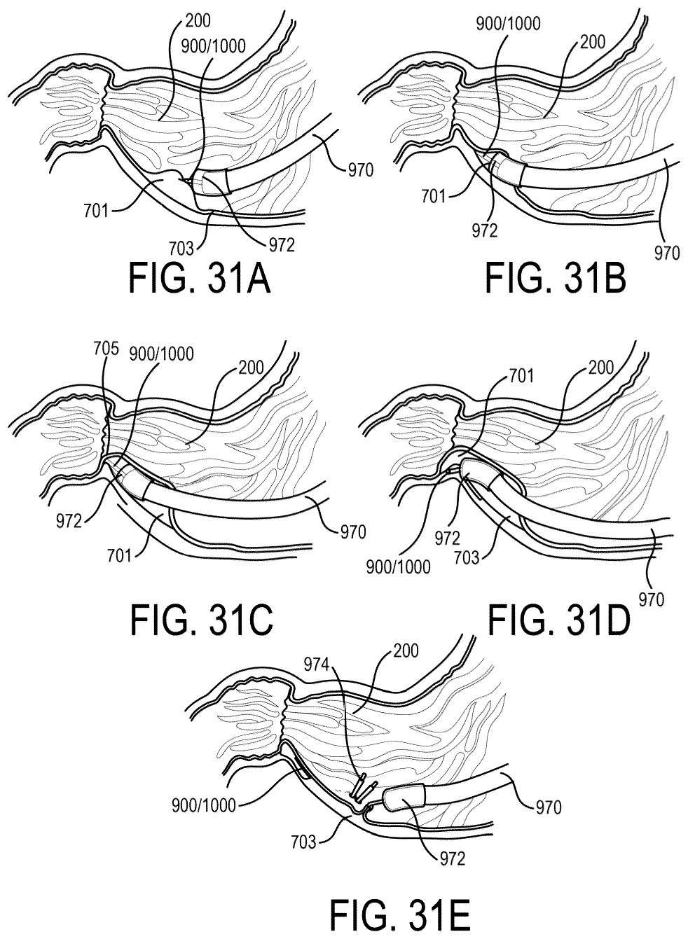

In one embodiment of a temporary GES stimulator 900 shown in FIG. 27, there is shown an electronics module 919, which comprises batteries 32, an encapsulant 918, a self-sealing port 916, directional electrodes 901/903, a microcontroller 24, a pulse generator module 36, an electrode switching module 40/42, and power regulation circuit 34. A detachable cable 92 enables programming and other functionality from an external device such as a programmer or controller 90 or 80.

Electronics module 919 may receive power from one or two of the batteries 32 (e.g., primary coin cells). In the embodiment shown in FIG. 27, two electrodes 901 and 903 are located on one side of temporary GES 900, which can be configured to provide directionality or beam steering capabilities for the delivery of stimulation pulses. A hard-wired cable 92 (e.g., the detachable cable 92 shown in FIG. 27) can be configured to provide communication data from a programming device 80 or 90 to the temporary GES 900.

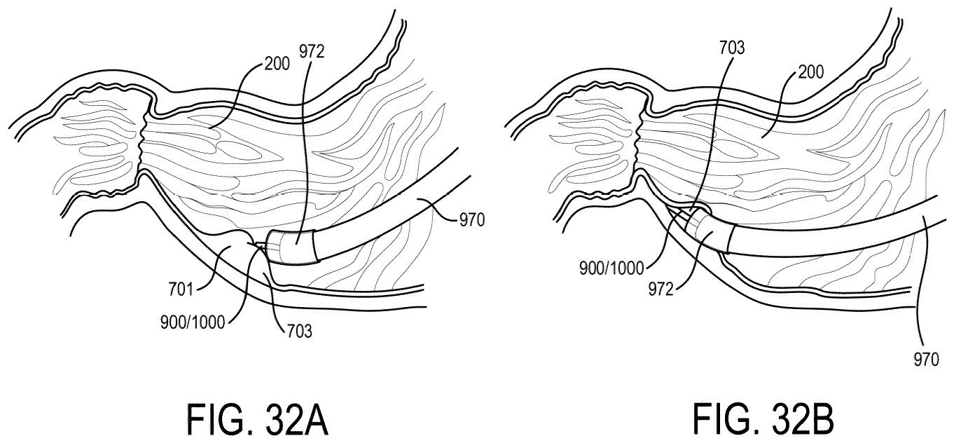

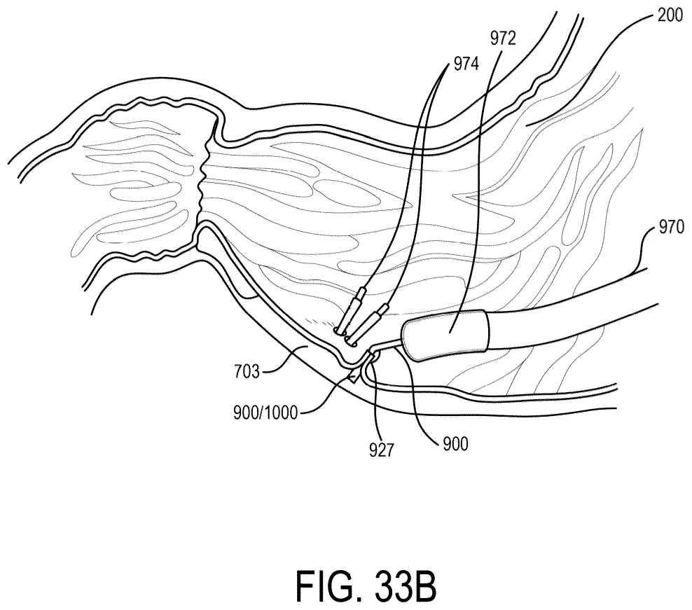

The assembly shown in FIG. 27 can be encapsulated in a polymer (e.g., silicone), which can provide insulation from body tissues and fluids. Cable 92 can be detached once temporary GES 900 has been positioned in and affixed to the stomach 200, and port 916 then self-seals (see, for example, FIGS. 27 and 28). In the embodiments illustrated in FIGS. 28 and 30, there are two or three annular electrodes 901, 903 and/or 905 that can be programmed such that at least one electrode acts as a cathode and the other electrode(s) acts as an anode, or vice versa. Other electrode configurations and numbers of electrodes are also contemplated for temporary GES 900.

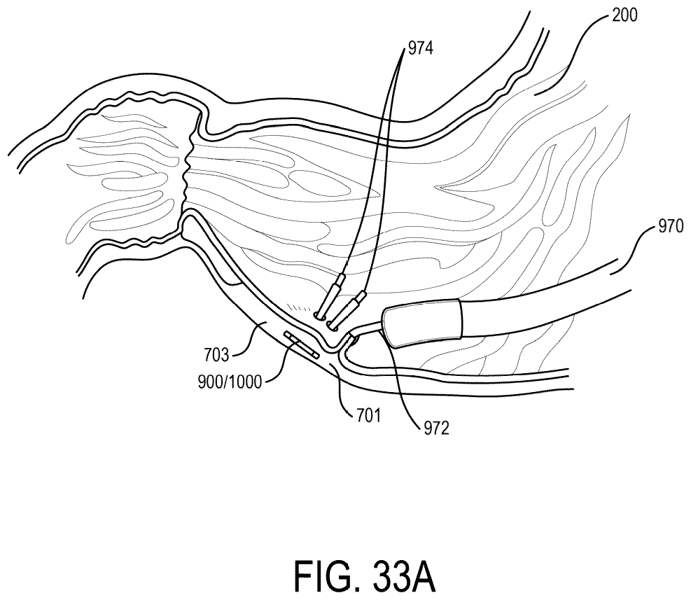

In another embodiment, communications between temporary GES 900 and controller/programmer 90 or 80 are wireless. A wireless communications module 94 (see FIG. 29) may include a low energy wireless Bluetooth (BLE) or other wireless communications circuit 93 and a wireless antenna 96. The wireless communications module may be located outside the patient's body. In one embodiment, an external subassembly is disconnected from the implant after the temporary GES 900 has been be set up and programmed.

In still another embodiment, three or more electrodes may be programmed to provide various different combinations of cathodes and anodes (see FIG. 28).

### Some Embodiments of Gastric Per Oral Endoscopic Myotomy (G POEM) Systems, Devices, Components, Techniques and Methods for Permanent or Temporary Gastric Electrical Stimulation

In some embodiments, and as described further below, as well as in the publication “Third-Space Endoscopy: The Final Frontier” to Hayat et al., Gastroenterology Report, 11, 2023, goac077 (which publication is hereby incorporated by reference into the specification of the present patent application, in its entirety), G POEM procedures, techniques and methods are adapted and configured to permit the implantation of a temporary gastric stimulator 900, or portion(s) thereof, in the submucosal layer of a patient's stomach 200 in the so-called “third space.” A related procedure that may also be employed is the G POP (Per-oral pyloromyotomy) procedure, more about which is said below.

In still other embodiments, as for example described in the publication “Gastric per-oral endoscopic myotomy: Indications, technique, outcomes, and future directions” to Bapaye et al., Int J Gastrointest Interv 2020; 9(2): 72-77 (which publication is hereby incorporated by reference into the specification of the present patent application, in its entirety), gastric per-oral endoscopic pyloromyotomy (G-POEM, POEP, POP) procedures, techniques and methods are adapted and configured to permit the implantation of a temporary gastric stimulator, or portion(s) thereof, in the submucosal layer of a patient's stomach 200 in the so-called “third space.”

In some embodiments, G POP and POP procedures, methods and techniques may also be employed to implant a temporary gastric stimulator or portions thereof into the third space. In some embodiments, G POP and/or POP procedures, techniques and methods G POP procedures are typically carried out via the lesser curve of the stomach, whereas G POEM procedures are usually carried out via the greater curve of the stomach.

According to some embodiments, in G POEM, G POP and POP procedures a submucosal tunnel to form a third space is formed about 5 cm from the pylorus, regardless of lesser or greater curve implantation approaches.

In other embodiments, surgical pyloromyotomy procedures and associated tunnelling of the submucosal layer to permit implantation of a temporary gastric stimulator or portions thereof in the third space is also contemplated.

According to the particular device and method embodiments that are to be used, conventional endoscopic devices and techniques also may be adapted to optimize third space tunnelling and formation for the purpose of implanting a temporary gastric stimulator (or portions thereof) therein.

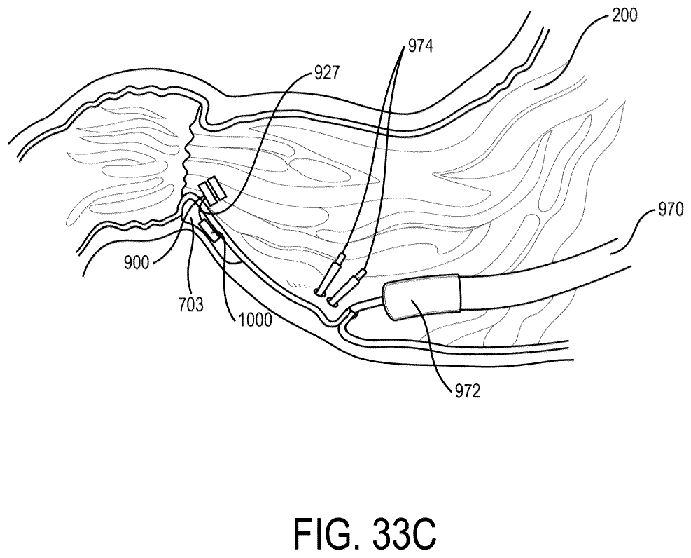

Third-space endoscopy is a novel, safe, and effective method for treating different gastrointestinal conditions by creating a submucosal tunnel. Illustrative steps of a Gastric Per Oral Endoscopic Myotomy (G POEM) procedure adapted for use in accordance with some embodiments of the implantation of a temporary gastric electrical stimulator 900, or portion(s) or components or elements thereof, are shown in FIGS. 31A through 31E.

As shown in FIG. 31A, an endoscope 970 is first used to create a saline bubble 701 in the submucosal layer 703 of the stomach. The distal end 972 of endoscope 970 is advanced into the newly created space (FIG. 31B) and temporary GES 900/1000 is placed therein. In one embodiment of a G POEM procedure, the endoscope 970 is advanced to the pylorus 705 (FIG. 31C), and the muscle is cut (FIG. 31D), thereby opening this valve. The endoscope 970 is then withdrawn, temporary GES 900/1000 is left behind in space 703, and the opening is closed using clips (FIG. 31E).

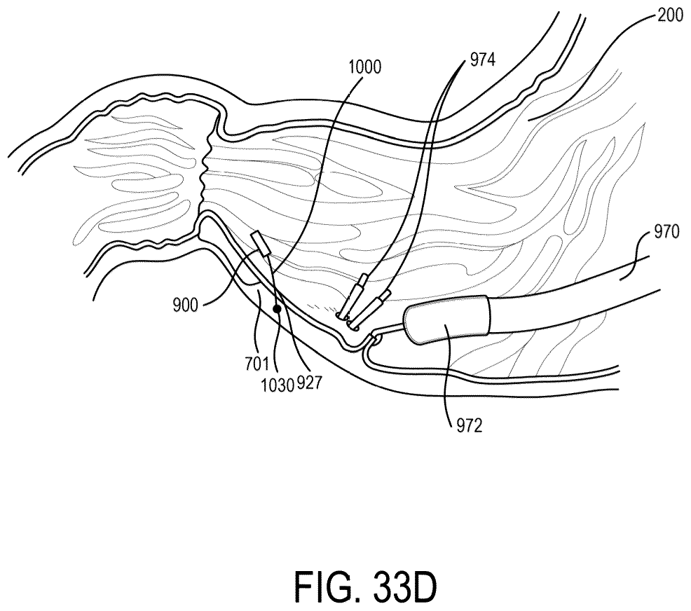

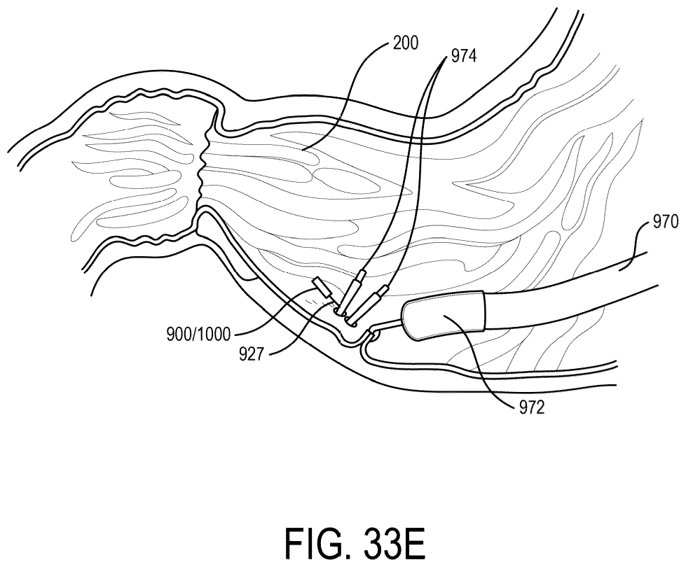

In another embodiment, and as shown in FIGS. 32A and 32B, a temporary GES 900/1000 is implanted in a similar manner to that shown above in FIGS. 31A-31E, using steps 31A and 31B thereof. At this point, the temporary gastric electrical stimulator 900/1000 can be inserted into the newly created tunnel and left in place. Stimulator 900/1000 may also be placed in the tunnel at the end of a G POEM procedure. See FIG. 33A.

In another embodiment, and as shown in FIG. 33B, a lead portion 1000 of temporary gastric electrical stimulator 900 is disposed within the newly created submucosal space, volume or pouch, while electronic capsule or stimulator module 900 is disposed outside the submucosal space, volume or pouch 703 inside stomach 200. If desired, device 900/1000 can be retrieved endoscopically at a later date by grasping capsule 900 with an endoscope and retracting it. Alternatively, a biodegradable link 927 disposed between capsule 900 and lead 100 may be provided that is configured to dissolve after a predetermined period of time has passed, and capsule 900 is released to pass through the patient's digestive tract. In another embodiment, a releasable link between the stimulator module or capsule 900 and the lead portion 1000 is provided, which upon receiving an instruction from a health care provider through wireless communication or after a predetermined period of time has passed following implantation, permits the stimulator module 900 to be released and pass through the patient's digestive tract.

In another embodiment, and as shown in FIG. 33C, a biodegradable link 927 between stimulator module 900 and lead portion 1000 dissolves over time, thereby releasing capsule 900 from lead portion 1000 to pass harmlessly through the digestive track.

In yet another embodiment, and as shown in FIG. 33D below, one or more biodegradable lead or stimulation module fixation features 1030, such as tines, dissolve over time, releasing the entire system 900 and 1000, or portion(s) thereof, to pass harmlessly through the digestive track.

In still another embodiment, the lead portion 1000 of the temporary gastric electrical stimulator 900 is fully biodegradable using bioabsorbable electronics, such as described in Wei Z, Xue Z, Guo Q., Recent Progress on Bioresorbable Passive Electronic Devices and Systems. Micromachines (Basel). 2021 May 22; 12(6): 600. doi: 10.3390/mi12060600. PMID: 34067419; PMCID: PMC8224698. In such an embodiment, the stimulator passes through the patient.

In a further another embodiment, and as shown below in FIG. 33E, a temporary gastric electrical simulator is affixed to the mucosal lining of the stomach using clips 974 without creating a tunnel, much as temporary gastric stimulation leads are affixed today. Such a design still has the advantage of not requiring a lead exiting from the patient's nose or mouth. Eventually, the clips release from the lining, allowing the system to pass through the patient. In still another embodiment, clips 974 are configured to be biodegradable. Biodegradable link 927 may optionally be provided between clips 974 and capsule/lead portion 900/1000.

Note that in some of the various embodiments of capsule/lead portion 900/1000 illustrated and described herein, lead portion/lead 1000 may not necessarily extend away from capsule 900 or be separate therefrom, and may be incorporated into capsule 900 as electrodes 901/903/905, for example.

As will now be seen, various embodiments of a neurostimulator or temporary gastric electrical stimulator are described above that are configured to be at least partially inserted into an endoscopically created submucosal space in the digestive tract. Various embodiments of methods of implanting such a stimulator comprise creating a submucosal “3^{rd} space” and inserting all or part of a neurostimulator or stimulator into the space are also described. In some embodiments, biodegradable portions of the neurostimulator or stimulator can be provided that are designed to release at least part of the stimulator, such as the lead portion, the stimulator module, and/or clips associated therewith, into the patient's digestive tract thereby allowing passage of the stimulator or portions thereof out of the patient.

Note that in some embodiments, while G POEM, POP, and related or similar endoscopic or surgical procedures may be employed to form a third space, tunnel or pouch configured to receive therein an implantable temporary gastric stimulator or portions thereof, muscles associated with the pylorus or other portions of the stomach may not be cut during such a procedure.

As will now be understood by those skilled in the art, some of the various embodiments described and disclosed herein permit a patient to undergo temporary gastric electrical stimulation prior to a permanent implant, without a wire running through the patient's nose or mouth for the duration of the trial. By placing all, or part, of the system within the created submucosal tunnel, stable fixation of at least portions of the stimulator is created. The stimulator can be retrieved via endoscope at the end of the trial. In the case of a system using a rechargeable or wirelessly powered device, the stimulator can be left inside the patient indefinitely, which cannot be done with wired systems in use today. If bioabsorbable fixation is used, the device can just pass through the patient. The patient is not required to come back to the office to have the lead or other portions of the stimulator removed.

What have been described above are examples and embodiments of the methods, systems, devices and components described and disclosed herein. It is, of course, not possible to describe every conceivable combination of components or methodologies for purposes of describing the invention, but one of ordinary skill in the art will recognize that many further combinations and permutations of the devices and methods described and disclosed herein are possible. For example, the various embodiments may employ external pulse generators or stimulators, or implantable pulse generators. They may employ permanent or temporary components, devices or systems. They may be implanted endoscopically or using other techniques. Leadless embodiments are contemplated, as are embodiments which are configured to operate in conjunction with external or implanted systems, devices or components such as sensors. The systems or devices may be wired or wireless. Charging of batteries in implanted devices may be accomplished inductively or transcutaneously.

In addition, although several embodiments of GES/IPG/capsule 900 are described and disclosed herein as being intended for implantable temporary gastric stimulation applications, in some embodiments GES/IPG/capsule 900 and any corresponding medical electrical lead(s) 1000 operably connected thereto may also be configured for implantable permanent gastric stimulation applications.

Accordingly, the devices and methods described and disclosed herein are intended to embrace all such alterations, modifications and variations that fall within the scope of the appended claims. In the claims, unless otherwise indicated, the article “a” is to refer to “one or more than one.”

The foregoing description and disclosure outline features of several embodiments so that those skilled in the art may better understand the detailed descriptions set forth herein. Those skilled in the art will now understand that many different permutations, combinations and variations of the systems, devices, components, methods, procedures and techniques described and disclosed herein fall within the scope of the various embodiments. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

After having read and understood the present specification, those skilled in the art will now understand and appreciate that the various embodiments described herein provide solutions to long-standing problems in the effective use of neurostimulation systems in gastric stimulation applications.

We claim:

1. A temporary or permanent gastric stimulator comprising a stimulation module and at least one stimulation electrode operably connected thereto and associated therewith, the stimulation module forming or forming a portion of an implantable pulse generator (IPG), wherein at least portions of the stimulator are configured to fit within a first space or volume formed in a submucosal layer of a patient's stomach using an endoscopic device, wherein the endoscopic device is configured to be advanced into the first space or volume to form a second space or tunnel in the submucosal layer or to form an extension of the first space in the submucosal layer, and further wherein at least portions of the stimulator are configured to be implanted in at least a portion of the second space or tunnel of the submucosal layer of the patient's stomach, or to be implanted in the extension of the first space of the submucosal layer of the patient's stomach.

2. The temporary or permanent gastric stimulator of claim 1, wherein the temporary or permanent gastric stimulator is further configured to, over a first predetermined period of time, generate first electrical stimulation signals in the IPG, the first electrical stimulation signals having one or more frequencies ranging between about 2 Hz and about 120 Hz, one or more pulse widths ranging between about 100 μsec. and about 10 msec., one or more amplitudes ranging between about 0.1 mA and about 20 mA.

3. The temporary or permanent gastric stimulator of claim 2, wherein the temporary or permanent gastric stimulator is further configured to provide, over at least a portion of the first predetermined period of time, the first electrical stimulation signals through at least one medical electrical lead and one or more electrodes thereof to a portion of the stomach of the patient, and over a second predetermined period of time, generate second electrical stimulation signals in the IPG, the second electrical stimulation signals having one or more of frequencies, pulse widths and amplitudes less than one or more of the corresponding frequencies, pulse widths and amplitudes of the first electrical stimulation signals.

4. The temporary or permanent gastric stimulator of claim 3, wherein the temporary or permanent gastric stimulator is further configured to provide, over at least a portion of the second predetermined period of time, the second electrical stimulation signals through the at least one medical electrical lead and the one or more electrodes thereof to the portion of the stomach of the patient.

5. The temporary or permanent gastric stimulator of claim 4, wherein the temporary or permanent gastric stimulator is further configured to provide, as one or more of the frequencies, pulse widths, and amplitudes of the electrical stimulation signals provided to the patient continue to be successively reduced, determining one or more of the frequency, pulse width, and amplitude parameters of the electrical stimulation signal wherein efficacy in treating the one or more gastrointestinal disorders of the patient is reduced or lost.

6. The temporary or permanent gastric stimulator of claim 5, wherein the temporary or permanent gastric stimulator is further configured to provide, on the basis of the frequency, pulse width, and amplitude parameters determined to be associated with reduced or lost efficacy in treating the one or more gastrointestinal disorders of the patient, generating and providing to the patient chronic electrical stimulation signals having one or more of increased frequency, pulse width, and amplitude parameters compared to the frequency, pulse width, and amplitude parameters determined to be associated with substantially reduced or lost efficacy in treating the one or more gastrointestinal disorders of the patient.

7. The temporary or permanent gastric stimulator of claim 6, wherein the one or more gastrointestinal disorders of the patient include at least one of nausea, vomiting, early satiety, postprandial fullness, and abdominal pain and the method results in more rapid and accurate programming and determination of gastric stimulation parameters for the patient compared to conventional gastric stimulation programming techniques or methods.

8. The temporary or permanent gastric stimulator of claim 1, wherein the implantable stimulation module and the at least one electrode are contained in, or each form a portion of, a capsule or housing.

9. The temporary or permanent gastric stimulator of claim 1, wherein the temporary or permanent gastric stimulator further comprises electrical stimulation electronics and a power source associated therewith.

10. The temporary or permanent gastric stimulator of claim 1, wherein the temporary or permanent gastric stimulator further comprises communication electronics configured to permit hard-wired or wireless communication and control or programming thereof from a programmer or controller.

11. The temporary or permanent gastric stimulator of claim 1, wherein the stimulation module comprises at least one return or ground electrode.

12. The temporary or permanent gastric stimulator of claim 1, wherein the at least one stimulation electrode forms a portion of a medical electrical lead, the medical electrical lead being operably connected to the implantable stimulation module and configured to deliver electrical stimulation signals from the module to the lead.

13. The temporary or permanent gastric stimulator of claim 12, wherein the lead comprises multiple stimulation electrodes.

14. The temporary or permanent gastric stimulator of claim 13, wherein the electrodes are at least one of unipolar, bipolar and multi-polar.

15. The temporary or permanent gastric stimulator of claim 13, wherein the lead comprises at least one return or ground electrode.

16. The temporary or permanent gastric stimulator of claim 13, wherein a biodegradable or releasable link is disposed between at least a portion of the lead and the stimulation module, and at least one of the lead and the stimulation module is configured to be released from attachment to or positioning within the tunnel, the first space, the second space, or the submucosal layer, and then to pass harmlessly through the patient's digestive system after the biodegradable link has dissolved or the link has been released after a predetermined period of time has passed or upon receipt of a command by the stimulator from an external communication device.

17. The temporary or permanent gastric stimulator of claim 13, wherein the temporary or permanent gastric stimulator, or one or more portions thereof such as a lead portion, comprises at least one fixation member or feature configured to affix the stimulator or portion thereof to the submucosal layer.

18. The temporary or permanent gastric stimulator of claim 17, wherein the fixation member or feature comprises one or multiple ones of a tine, a helical fixation wire, a staple, and a fixation pin.

19. The temporary or permanent gastric stimulator of claim 1, wherein the temporary or permanent gastric stimulator further comprises one or more of a rechargeable battery, a primary battery, and a power source.

20. The temporary or permanent gastric stimulator of claim 1, wherein the stimulator is implanted using one or more G POEM steps, techniques or methods.

21. The temporary or permanent gastric stimulator of claim 1, further comprising the temporary or permanent gastric stimulator or one or more portions thereof, being configured to be passed safely through the patient's digestive tract after being released from the stomach by the temporary or permanent gastric stimulator or one or more portions thereof being released or through the action of a biodegradable link dissolving.

22. A method of implanting a temporary or permanent gastric stimulator in a stomach of a patient, the temporary or permanent gastric stimulator comprising a stimulation module and at least one stimulation electrode operably connected thereto and associated therewith, the stimulation module forming or forming a portion of an implantable pulse generator (IPG), wherein at least portions of the stimulator are configured to fit within a first space or volume formed in a submucosal layer of a patient's stomach using an endoscopic device, wherein the endoscopic device is configured to be advanced into the first space or volume to form a second space or tunnel in the submucosal layer or to form an extension of the first space in the submucosal layer, and further wherein at least portions of the stimulator are configured to be implanted in at least a portion of the second space or tunnel of the submucosal layer of the patient's stomach, or to be implanted in the extension of the first space of the submucosal layer of the patient's stomach, the method comprising:
(a) advancing the endoscopic device into the first space or volume to form the second space or tunnel in the submucosal layer or to form an extension of the first space in the submucosal layer, and
(b) using the endoscopic device, implanting the stimulator in at least a portion of the second space or tunnel of the submucosal layer of the patient's stomach or implanting the stimulator in the extension of the first space of the submucosal layer of the patient's stomach.

23. The method of claim 22, further comprising the temporary or permanent gastric stimulator generating over a first predetermined period of time first electrical stimulation signals, the first electrical stimulation signals having one or more frequencies ranging between about 2 Hz and about 120 Hz, one or more pulse widths ranging between about 100 μsec. and about 10 msec., and one or more amplitudes ranging between about 0.1 mA and about 20 mA.

24. The method of claim 23, wherein the temporary or permanent gastric stimulator further provides, over at least a portion of the first predetermined period of time, the first electrical stimulation signals through at least one medical electrical lead and one or more electrodes thereof to a portion of the stomach of the patient, and over a second predetermined period of time, generates second electrical stimulation signals in the IPG, the second electrical stimulation signals having one or more of frequencies, pulse widths and amplitudes less than one or more of the corresponding frequencies, pulse widths and amplitudes of the first electrical stimulation signals.

25. The method of claim 24, wherein the temporary or permanent gastric stimulator further provides, over at least a portion of the second predetermined period of time, the second electrical stimulation signals through the at least one medical electrical lead and the one or more electrodes thereof to the portion of the stomach of the patient.

26. The method of claim 25, wherein the temporary or permanent gastric stimulator further provides, as one or more of the frequencies, pulse widths, and amplitudes of the electrical stimulation signals provided to the patient continues to be successively reduced, determining one or more of the frequency, pulse width, and amplitude parameters of the electrical stimulation signal wherein efficacy in treating the one or more gastrointestinal disorders of the patient is reduced or lost.

27. The method of claim 26, wherein the temporary or permanent gastric stimulator further provides, on the basis of the frequency, pulse width, and amplitude parameters determined to be associated with reduced or lost efficacy in treating the one or more gastrointestinal disorders of the patient, generating and providing to the patient chronic electrical stimulation signals having one or more of increased frequency, pulse width, and amplitude parameters compared to the frequency, pulse width, and amplitude parameters determined to be associated with substantially reduced or lost efficacy in treating the one or more gastrointestinal disorders of the patient.

28. The method of claim 27, wherein the one or more gastrointestinal disorders of the patient include at least one of nausea, vomiting, early satiety, postprandial fullness, and abdominal pain and the method results in more rapid and accurate programming and determination of gastric stimulation parameters for the patient compared to conventional gastric stimulation programming techniques or methods.

29. The method of claim 22, wherein the implantable stimulation module and the at least one electrode are contained in, or each form a portion of, a capsule or housing.

30. The method of claim 22, wherein the temporary or permanent gastric stimulator further comprises electrical stimulation electronics and a power source associated therewith.

31. The method of claim 22, wherein the temporary or permanent gastric stimulator further comprises communication electronics configured to permit hard-wired or wireless communication and control or programming thereof from a programmer or controller.

32. The method of claim 22, wherein the stimulation module comprises at least one return or ground electrode.

33. The method of claim 22, wherein the at least one stimulation electrode forms a portion of a medical electrical lead, the medical electrical lead being operably connected to the implantable stimulation module and configured to deliver electrical stimulation signals from the module to the lead.

34. The method of claim 33, wherein the lead comprises multiple stimulation electrodes.

35. The method of claim 34, wherein the electrodes are at least one of unipolar, bipolar and multi-polar.

36. The method of claim 34, wherein the lead comprises at least one return or ground electrode.

37. The method of claim 34, wherein a biodegradable or releasable link is disposed between at least a portion of the lead and the stimulation module, and at least one of the lead and the stimulation module is configured to be released from attachment to or positioning within the tunnel, the first space, the second space, or the submucosal layer, and then to pass harmlessly through the patient's digestive system after the biodegradable link has dissolved or the link has been released after a predetermined period of time has passed or upon receipt of a command by the stimulator from an external communication device.

38. The method of claim 34, wherein the temporary or permanent gastric stimulator, or one or more portions thereof such as a lead portion, comprises at least one fixation member or feature configured to affix the stimulator or portion thereof to the submucosal layer.

39. The method of claim 38, wherein the fixation member or feature comprises one or multiple ones of a tine, a helical fixation wire, a staple, and a fixation pin.

40. The method of claim 22, wherein the temporary or permanent gastric stimulator further comprises one or more of a rechargeable battery, a primary battery, and a power source.

41. The method of claim 22, wherein the stimulator is implanted using one or more G POEM steps, techniques or methods.

42. The method of claim 22, further comprising the temporary or permanent gastric stimulator or one or more portions thereof, being configured to be passed safely through the patient's digestive tract after being released from the stomach by the temporary or permanent gastric stimulator or one or more portions thereof being released or through the action of a biodegradable link dissolving.
